(19) United States
(12) Patent Application Publication
(10) Pub. No.: US 2017/0023551 A1
(43) Pub. Date: Jan. 26, 2017

(54) Tools and Methods for Targeting Oligonucleotide Repeat RNA Toxicity

(71) Applicant: The General Hospital Corporation, Boston, MA (US)

(72) Inventors: Gary B. Ruvkun, Newton, MA (US); Susana Garcia, Boston, MA (US)

(21) Appl. No.: 15/215,197

(22) Filed: Jul. 20, 2016

Related U.S. Application Data
(60) Provisional application No. 62/194,420, filed on Jul. 20, 2015.

(51) Int. Cl. G01N 33/50; A01K 67/033; C12N 15/113; C12Q 1/68

(52) U.S. Cl. CPC G01N 33/5085; C12Q 1/6897; A01K 67/0336; C12N 15/1137; G01N 2333/43534; A01K 2217/203; A01K 2227/703; A01K 2267/0318; C12N 2310/14; C12N 2330/31

(57) ABSTRACT

Described are Caenorhabditis elegans (C. elegans) strains exhibiting an RNA toxicity phenotype. The C. elegans strains comprise a detectable reporter gene expressed in one or more cell types, with the expressed reporter gene RNA having in instance of at least fifty oligonucleotide repeats (e.g., trinucleotide repeats). Exemplary C. elegans reporter strains are generated that exhibit phenotypes characteristic of the human disorder Myotonic Dystrophy 1. The C. elegans strains are amenable for high-throughput screening applications, for both gene target as well as small molecule identification.

### CLAIM OF PRIORITY

This application claims priority under 35 USC §119(e) to U.S. Patent Application Ser. No. 62/194,420, filed on Jul. 20, 2015. The entire contents of the foregoing are hereby incorporated by reference.

### FEDERALLY SPONSORED RESEARCH OR DEVELOPMENT

This invention was made with government support under Grant No. AG043184 awarded by the National Institutes of Health. The government has certain rights in the invention.

### FIELD OF THE INVENTION

The invention in various aspects relates to tools and methods for elucidating the biology of nucleotide repeat RNA toxicity, as well as to the identification of molecular targets and preparation of pharmaceutical agents useful for treating such conditions.

### BACKGROUND

Expansions in nucleotide repeat sequences cause many neuromuscular degenerative disorders and can occur in noncoding as well as coding regions of genes. For example, expansions of CTG repeats in the 3′ untranslated region (3′UTR) of the DMPK protein kinase gene causes myotonic dystrophy 1 (DM1), an autosomal dominant degenerative disease. DM1 CTG expansions range up to >2,000 repeats, while normal CTG lengths range from 5-36 repeats. RNA toxicity is the cause of DM1 pathology, where transcripts containing expanded CUG repeats accumulate in the nucleus as discrete RNA foci. The length of repeat expansion correlates with DM1 disease onset and severity. Expanded CUG repeat RNA transcripts disrupt alternative RNA splicing mediated by muscleblind-like (MBNL) and the CUG binding protein 1 (CUG-BP1) RNA binding protein families, causing toxicity. However, disruption of these splicing factors, in particular of MBNL, does not explain the many phenotypes observed in DM disorders. There are believed to be additional unknown factors and mechanisms in expanded CUG repeat pathogenesis.

An object of the invention is to provide tools for elucidating the biology of nucleotide repeat RNA toxicity, including tools for identifying factors and mechanisms behind nucleotide repeat pathogenesis, and tools for screening candidate therapeutics agents. It is a further object of the invention to provide methods for selecting candidate agents, and preparing such agents for therapeutic use.

Other objects of the invention will be apparent from the following description of the invention.

### SUMMARY OF THE INVENTION

In some aspects, the invention provides Caenorhabditis elegans (C. elegans) strains exhibiting an RNA toxicity phenotype. The C. elegans strain comprises a detectable reporter gene expressed in one or more cell types, with the expressed reporter gene RNA having in instance of at least fifty oligonucleotide repeats (e.g., trinucleotide repeats). The C. elegans strains described herein are amenable for high-throughput screening applications, for both gene target as well as small molecule identification.

Exemplary C. elegans reporter strains were generated that exhibit phenotypes characteristic of the human disorder Myotonic Dystrophy 1. Myotonic Dystrophy 1 (DM1) is a neuromuscular disease caused by expansions in a CUG repeat in the 3′UTR of a protein kinase gene. In these reporter strains, C. elegans muscle cells expressed a gene coding for green fluorescent protein (GFP) followed by CUG repeat expansions in its 3′UTR. These strains recapitulated many of the characteristic DM1 disease-associated phenotypes, such as muscle dysfunction and accumulation of RNA nuclear foci containing expanded CUG transcripts. These animals were used in the identification of genes previously not known to be implicated in myotonic dystrophy and can further contribute to uncover the full complement of genes that regulate DM1 toxicity. The genes identified can be used as therapeutic targets. Further, because these reporter strains exhibit DM1 toxicity phenotypes they are ideal for the identification of compounds/small molecules that can be used as novel therapeutic approaches for DM1, or other RNA-associated disorders.

Analysis of C. elegans muscle function defects caused by expanded CUG repeats, together with cell biological analysis of these aberrant RNAs in wild type and in a library of gene-inactivated backgrounds, identified gene inactivations that modify expanded CUG repeat toxicity and CUG repeat foci accumulation, the hallmark of DM disorders. These modifiers of expanded CUG repeat toxicity include the nonsense-mediated mRNA decay (NMD) pathway, which targets CUG repeat-containing transcripts for degradation. NMD regulation of CUG repeat foci accumulation is a conserved mechanism present in both C. elegans and human cells. Recognition of these CUG repeat-containing transcripts for degradation by NMD is dependent on repeat-sequence composition.

Thus, in some embodiments, the C. elegans strain exhibits DM1 toxic phenotypes. These strains are of particular interest in the neuromuscular degenerative repeat-associated field because they share molecular and cellular characteristics, including loss of muscle function, with RNA-associated neuromuscular degenerative disorders, such as fragile X syndrome, amyotrophic lateral sclerosis, spinocerebellar ataxia 2, 3, 8, 10 and 12, etc. These animals allow for high-throughput screening and identification of novel genetic modifiers of RNA-repeat toxicity.

Further, the loss of locomotion observed in these animals, due to the expression of toxic RNAs, makes these strains uniquely amenable to both forward and reverse genetics for gene identification. This approach will identify new genes that can be used for drug therapy in RNA disorders in general and myotonic dystrophies, in particular. These approaches will also provide a better understanding of the pathways that regulate RNA-based toxic mechanisms.

This, provided herein are Caenorhabditis elegans (C. elegans) strains exhibiting an RNA toxicity phenotype, the strains comprising a detectable reporter gene expressed in one or more cell types, the expressed reporter gene RNA having in instance of at least fifty oligonucleotide repeats.

In some embodiments, the oligonucleotide repeats are repeats of from 3 to 6 nucleotides, e.g., trinucleotide repeats.

In some embodiments, the detectable reporter gene is stably integrated into the C. elegans genome.

In some embodiments, the C. elegans exhibits a decline in adult stage reporter gene protein levels.

In some embodiments, the reporter gene RNA accumulates into nuclear foci.

In some embodiments, the reporter gene is expressed from a tissue-specific promoter.

In some embodiments, the reporter gene is expressed in body wall muscle cells or in neurons.

In some embodiments, the C. elegans displays a motor defect in the adult stage.

In some embodiments, the detectable reporter gene encodes a fluorescent or luminescent protein.

In some embodiments, the detectable reporter gene encodes a green fluorescent protein (GFP).

In some embodiments, the oligonucleotide repeats are in the 3′ UTR of the detectable reporter gene.

In some embodiments, the repeats are trinucleotide repeats that encode polyglutamine. In some embodiments, the repeats are trinucleotide repeats of CUG. In some embodiments, the repeats are trinucleotide repeats of CGG or CAG.

In some embodiments, the reporter gene RNA has at least 70 repeats of the oligonucleotide, at least 100 repeats of the oligonucleotide, or at least 120 repeats of the oligonucleotide.

In some embodiments, the C. elegans strain further comprises an inactivation, overexpression, or modification of at least one endogenous gene. In some embodiments, the C. elegans strain comprises an inactivation of at least one endogenous gene by RNAi.

In some embodiments, the endogenous gene encodes a signaling protein, a protein involved in RNA processing or degradation, RNA transport, transcription, DNA repair or recombination, or translation.

In some embodiments, the endogenous gene encodes a protein of the nonsense-mediated mRNA decay pathway.

In some embodiments, the endogenous gene is a gene listed in Table 2 or 3.

Also provided herein are multiwell plates comprising a C. elegans strain as described herein in one or more, e.g., each, of a plurality of wells.

In some embodiments, the multiwell plates comprise at least one well containing a C. elegans strain that does not exhibit an RNA toxicity phenotype, e.g., at least one C elegans strain that does not exhibit an RNA toxicity phenotype and that has a non-pathogenic amount of oligonucleotide repeats.

In some embodiments, the multiwell plates comprise from ten to twenty C. elegans organisms per well.

Also provided herein are methods for identifying an agent that modulates an RNA toxicity phenotype, comprising: providing a multiwell plate as described herein, adding a candidate agent to each of a plurality of wells, and quantifying an effect on said RNA toxicity phenotype.

In some embodiments, the effect on said RNA toxicity phenotype is quantified by the level of protein expression of said reporter gene and/or cellular location of the reporter gene RNA.

In some embodiments, the effect on said RNA toxicity phenotype is quantified by the accumulation of RNA into nuclear foci.

In some embodiments, the methods include quantifying a change in motility.

In some embodiments, the methods include selecting an agent that reduces said RNA toxicity phenotype.

Also provided herein are methods for making a pharmaceutical composition for treatment of a condition associated with RNA toxicity, the method comprising identifying an agent using a method described herein, and formulating said agent as a pharmaceutically acceptable composition.

In some embodiments, the agent is formulated for systemic administration.

In some embodiments, the agent inhibits the expression or activity of a gene selected from Table 2 or 3.

In some embodiments, the agent increases the expression or activity of a gene selected from Table 2 or 3.

In some embodiments, the gene is involved in the nonsense-mediated mRNA decay pathway.

Also provided herein are methods for treating a condition characterized by RNA toxicity, comprising administering a pharmaceutical composition prepared according to a method described herein to a patient in need. In some embodiments, the condition is myotonic dystrophy 1 (DM1), Fragile X syndrome, Huntington's disease-like 2, spinocerebellar ataxia, or amyotrophic lateral sclerosis.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other aspects and embodiments of the invention will be apparent from the following detailed description.

### DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-G show expanded CUG-dependent C. elegans muscle phenotypes. FIG. 1A provides a diagram of CUG-containing plasmids for expression in C. elegans muscle cells, under the myo-3 promoter. n indicates number of CUG repeats. FIG. 1B depicts quantification of GFP expression levels from reporter genes with 123 CUG repeats or 0 CUG repeats in the 3′UTR, relative to actin. Graph shows mean and s.d. for 3 independent experiments, p was determined by Student's t test. Bottom shows western blots using GFP and actin antibodies, actin was used for sample normalization. FIG. 1C depicts motility assays for 6d adults. Data plotted corresponds to average percentage of population to reach food at each time point. Error bars represent SD from at least 3 independent experiments; in each experiment, 3-5 replicas of ca. 100-150 animals were analyzed. FIG. 1D shows confocal single molecule RNA fluorescence in situ hybridization (SM-FISH) images of C. elegans muscle cells for GFP RNA transcripts (right, white); nucleus are stained with DAPI. Arrows indicate expanded CUG nuclear foci, and the asterisk () indicates the nucleolus. FIG. 1E shows computational analysis of SM-FISH muscle cell images of 0CUG, 8CUG and 123 CUG animals. Each dot corresponds to an analyzed SM-FISH image. The dotted square indicates the region of clustering of the 123 CUG images (solid dots). FIG. 1F shows confocal SM-FISH images of C. elegans muscle cells for GFP RNA transcripts (right, white); nucleus as stained with DAPI and mCherry fluorescence is shown on the right. The strains express GFP with 123CUG or 0CUG in a mCHERRY or MBL-1::mCHERRY backgrounds. Arrows indicate expanded CUG nuclear foci. MBL-1::mCHERRY localizes to the nucleus. FIG. 1G shows computational analysis of SM-FISH images of 0CUG, 0CUG;mbl-1::mCherry, 123CUG and 123CUG;mbl-1::mCherry animals.

FIGS. 2A-B depict identification of gene inactivation that modulates expanded CUG repeat toxicity. FIG. 2A shows gene inactivations that disrupt the late stage down-regulation of GFP fluorescence mediated by 123 CUG repeats in the 3′ UTR. Fluorescent microscopy images of the strains 123CUG and the control 0CUG, on different RNAi gene inactivations: empty vector control (ctrl), npp-4, hda-1, C06A1.6 and smg-2. Images were taken at the 3d old adult stage. Bar, 200 μm. FIG. 2B shows genetic suppressors and enhancers of expanded CUG repeat toxicity. Graph of velocity measurements of 0CUG (grey) and 123CUG (white) animals fed on different gene inactivations. The plotted velocities (μm/sec) correspond to the median of at least two experiments, where the red bars correspond to strains fed on control vector. Red line indicates the median velocity, and white shading represents the 25^{th} and 75^{th} percentile for the 123CUG animals fed on control vector. The dotted orange line represents the maximum and minimum of the median velocity for 123CUG animals fed on control vector. Indicated by red asterisk (*) are the significant gene inactivations, where significance was determined by Kolmogorov-Smirnov p-value. The black asterisk indicates the gene smg-2.

FIGS. 3A-C show suppressors and enhancers of expanded CUG toxicity affect nuclear foci. FIG. 3A shows confocal SM-FISH images of GFP RNA transcripts (white), DAPI stained nucleus and merge of C. elegans muscle cells. Shown are 123CUG and the 0CUG control in different RNAi gene inactivations: empty vector control (ctrl), C06A1.6 and npp-4. Arrows indicate expanded CUG nuclear foci. FIG. 3B shows computational analysis of SM-FISH images of 123CUG animals with different gene inactivations and control (ctrl). Results are plotted as bar graphs were gene inactivations corresponding to bars on the right of the control exhibit an increase in detected foci area, and conversely bars on the left of the control exhibit a decrease in foci area, relative to the control. The cfim-2 and F48E8.6 gene inactivations are similar to ctrl. FIG. 3C shows C. elegans muscle cells confocal SM-FISH images of GFP RNA transcripts (white), DAPI stained nucleus, merge of GFP RNA and nucleus images, and mCherry translational fusion protein. Strains imaged are 123CUG and 0CUG animals, in a mCHERRY (control) or NPP-4::mCHERRY backgrounds. Arrows indicate expanded CUG nuclear foci.

FIGS. 4A-D show that the NMD pathway modulates expanded CUG transcripts degradation and nuclear foci accumulation. FIG. 4A shows fluorescent microscopy images of 2d old adult animals expressing either 123 CUG repeats or 0CUG in the backgrounds: wild type (wt), smg-2(qd101), smg-1(r861) and smg-6(r896). Scale bars correspond to 200 μm. FIG. 4B depicts qRT-PCR assay for gfp levels in animals expressing either 123 CUG repeats or the control GFP in different backgrounds: wild type (wt), smg-2(qd101), smg-1(r861) and smg-6(r896). Wild type=1.0. Error bars represent SEM for three biological replicates. FIG. 4C shows confocal SM-FISH images of GFP RNA transcripts (white), DAPI stained nucleus and merge of C. elegans muscle cells. The strains imaged are 123 CUG and 0CUG animals, in wild type (wt) and smg-2(qd101). Arrows indicate expanded CUG nuclear foci. FIG. 4D shows computational analysis of SM-FISH images of 0CUG, 0CUG in smg mutant backgrounds, 123 CUG and 123 CUG in smg mutant backgrounds.

FIG. 5 shows that 3′UTR CUG repeat sequence composition triggers NMD recognition for degradation. Fluorescent microscopy images of the strains 123 CUG, GC-rich and AT-rich, in different RNAi gene inactivations: empty vector control (ctrl), smg-1, smg-2, and smg-6. Images of 3d old adult animals. Bar, 200 μm.

FIGS. 6A-B show NMD downregulation causes an increase in CUG repeat mRNA foci number in myotonic dystrophy 1 patient fibroblast cells. FIG. 6A shows SM-FISH of DM1-affected or normal human fibroblast cells in which UPF1 was downregulated relative to control non-transfected or transfected with scrambled siRNAs (mock) cells. The DM1 human fibroblast cell line used expressed the gene dmpk bearing 2000CUG in its 3′UTR. FIG. 6B provides a histogram which represents the distribution of the number of foci in DM1 cells that were downregulated for UPF1, mock and non-transfected controls. UPF1 downregulation led to a significant increase in the number of nuclear foci present relative to mock (p<0.0001) and non-transfected cells (p<0.00003), using t-student test. N indicates the total number of cells analyzed. Two independent experiments were performed. Bar, 5 μm.

FIGS. 7A-E shows that C. elegans expressing expanded CUG repeats exhibit locomotion defects. FIG. 7A depicts a representation of motility assays performed using agar plates containing an E. coli food ring. The food ring had a 2 cm radius. FIG. 7B depicts motility assays for 2d adults. Data plotted corresponds to the average percentage of population to reach the food at each time point. Error bars represent SD from at least 3 independent experiments; in each experiment, 3-5 replicas of ca. 100-150 animals were analyzed. FIGS. 7C-E show computational analysis of SM-FISH images. FIG. 7C shows that analysis starts with computational identification of the nuclear region based on DAPI staining in an SM-FISH image of a 123CUG animal. Following nucleus identification, FIG. 7D shows that there is computational delineation of cytoplasmic versus nuclear spaces in the SM-FISH image corresponding to the GFP RNA transcript probes. FIG. 7E shows analysis of pixel intensities for each SM-FISH image, corresponding to low RNA, high RNA densities and RNA foci in both the nucleus and cytoplasm.

FIGS. 8A-F show that expression of MBL-1::mCherry in C. elegans muscle cells increases expanded CUG transcript recruitment and mutant transcript nuclear foci accumulation. Schematic drawing of the MBL-1::mCHERRY construct (FIG. 8A), and C. elegans body wall muscle cells (FIG. 8B). FIG. 8C shows MBL-1::mCHERRY exhibits a diffuse cellular distribution with nuclear accumulation. FIG. 8D shows C. elegans muscle cells confocal SM-FISH images of GFP RNA transcripts (white), DAPI stained nucleus, merge of GFP RNA and nucleus images, and mCherry translational fusion protein. The muscle cells imaged correspond to animals expressing 123 CUG repeats and 0CUG, in a mCHERRY (control) or MBL-1::mCHERRY backgrounds. Arrows indicate expanded CUG nuclear foci. MBL-1::mCHERRY localizes to the nucleus. FIG. 8E provides genetic mosaic analysis of GFP intensity shows that GFP fluorescence, from 123 CUG mRNA transcripts, absent in cells expressing mbl-1::mCherry, relative to neighboring cells that fail to express mbl-1::mCherry. GFP fluorescence is not affected in the 0CUG control animals expressing mbl-1::mCherry. FIG. 8F provides confocal SM-FISH images of GFP RNA transcripts (white), DAPI stained nucleus, and merge of C. elegans muscle cells. The strains imaged were 123CUG and 0CUG in: empty vector control (ctrl) and mbl-1 gene inactivations. Arrows indicate expanded CUG nuclear foci.

FIGS. 9A-B show a screen approach for the identification of modulators of expanded CUG toxicity. FIG. 9A provides a representation of RNAi screen steps in the identification of modulators of expanded CUG repeat pathogenesis. FIG. 9B provides fluorescent microscopy images of the strains 123CUG and the control 0CUG, on different RNAi gene inactivations: empty vector control (ctrl), mbl-1 and aly-3. Images were taken at the 3d old adult stage. Bar, 200 μm.

FIG. 10 shows that suppressors and enhancers of expanded CUG toxicity have distinct effects on expanded CUG nuclear foci accumulation. Confocal SM-FISH images of GFP RNA transcripts (white), DAPI stained nucleus and merge of C. elegans muscle cells. The strains imaged were 123CUG and the control 0CUG, in different RNAi gene inactivations: empty vector control (ctrl), C06A1.6, str-67, mrt-2, npp-4 and smg-2. Arrows indicate expanded CUG nuclear foci.

FIG. 11 shows that gene inactivations have different effects on foci accumulation in the nucleus. Computational analysis of SM-FISH images of 0CUG animals, control, and 123CUG animals fed different gene inactivations and control vector. Each ‘dot’ shown in the graph represents one analyzed SM-FISH image, corresponding to a single imaged cell. The dotted square indicates the region of clustering of the samples corresponding to 123CUG animals on control vector. Labeled on the graph on the left, above the box, are the gene inactivations that cause an increase in bright pixel intensity, corresponding to an increase in foci size or number, relative to the 123CUG on control. The ‘grouping’ of 123CUG npp-4 inactivations in the upper right corner of the graph indicates both an increase in nuclear foci and in nuclear ‘single’ transcript localization relative to the 0CUG npp-4 controls that localize further to the left in the graph. The inset section displayed shows gene inactivations that cause a decrease in bright pixel intensity, relative to the 123CUG on control vector, corresponding to a decrease in foci size or number.

FIGS. 12A-B show that modulators of expanded CUG foci accumulate in the nucleus. Figure A shows over-expression of expanded CUG repeat suppressors caused a decrease in expanded CUG nuclear foci accumulation. C. elegans muscle cells confocal SM-FISH images of GFP RNA transcripts (white), DAPI stained nucleus, merge of GFP RNA and nucleus images, and mCherry translational fusion protein. The strains imaged are animals expressing 123CUG repeats and 0CUG in the following transgenic backgrounds: mCHERRY, NPP-4::mCHERRY, ASD-1::mCHERRY and RNP-2::mCHERRY. RNP-2 corresponds to the U1 small nuclear ribonucleoprotein A, and RNP-2::mCherry exhibits nuclear localization in C. elegans muscle cells. Figure B shows mutants in the NMD pathway cause an increase in expanded CUG nuclear foci accumulation. Confocal SM-FISH images of GFP RNA transcripts (white), DAPI stained nucleus and merge of C. elegans muscle cells. The strains imaged were animals expressing 123 CUG repeats and 0CUG, in the following backgrounds: wild type (wt), smg-1(r861) and smg-6(r896). Arrows indicate expanded CUG nuclear foci.

FIGS. 13A-D shows that NMD recognizes and degrades transcripts bearing GC-rich 3′UTRs. FIGS. 13A-B show that sequence composition of CUG repeat sequences in the 3′UTR contributes to NMD transcript recognition for degradation. FIG. 13A provides a schematic drawing of the GC-rich or AT-rich plasmids for expressions in C. elegans muscle cells. Figure B provides fluorescent microscopy images of strains expressing a GFP with a 300 bp ‘artificial’ insert in their 3′UTR containing the following GC percentages: 31%, 32%, 60% and 70%. Also included are the control strains containing 3′UTR inserts cloned from A. thaliana (34% GC) and P. aeruginosa (66% GC). These strains are shown in a wt background and in the background of the following smg mutants: smg-1(5861), smg-2(qd101) and smg-6(r896). The ‘fluorescence’ observed in the 60% GC and 70% GC strains in a wild type background corresponded to the characteristic gut autofluorescence, and no GFP signal was observed in the body wall muscle cells of these animals. Images were taken of animals at the L4 stage. Bar, 100 μm. FIGS. 13C-D show Western blot analysis of UPF1 down-regulation (24 hours post-transfection) by siRNA pool of unaffected (FIG. 13C) and DM1 (FIG. 13D) fibroblast cells, using UPF1-specific antibody. Fibroblasts showed a decrease of 40% in UPF1 levels relative to cells transfected with scrambled siRNAs (mock cells) in both unaffected (FIG. 13C) as well as DM1 (FIG. 13D) cells. GAPDH levels were used for normalization across samples.

FIGS. 14A-B provide a model of regulation of expanded RNAs. FIG. 14A shows a model for regulation of expanded RNA toxicity by the NMD pathway: NMD targets expanded CUG repeat transcripts for degradation reducing the levels of toxic RNAs present in the cells. A decrease in NMD function results in accumulation of toxic transcripts with increase in nuclear RNA foci and increase in toxicity with loss of motility. FIG. 14B shows a model for regulation of expanded RNA foci accumulation by the modulators of RNA toxicity identified: different pathways regulate expanded CUG repeat toxicity; an increase in foci causes a decrease in locomotion however, a decrease in foci doesn't necessarily correlate with a decrease in muscle toxicity.

### DETAILED DESCRIPTION OF THE INVENTION

The animals and methods described herein provide a unique capability for exploring the biology of, and potential therapies for, RNA toxicity disease. Unlike the majority of conventional drug screening that is carried out using cell free assays or in cell cultures containing limited cell types in relative isolation, C. elegans whole animal models copy the complexity underlying many diseases that is the result of a network of molecular crosstalk between multiple cell types, tissues, and organs. Unlike mice, hermaphroditic C. elegans has a generation time of only three days and a single animal can produce 300 genetically identical progeny, which enables extremely rapid and inexpensive propagation of millions of clonal animals. In these aspects, the invention simplifies the drug discovery and target identification process using C. elegans whole animal assays. These nematodes fit comfortably in standard 384- and even 1536-well assay plates and can be cultured in liquid making them amenable to HTS platforms. In addition, C. elegans are transparent enabling the use of fluorescent probes and reporters to visualize different organ systems and subcellular structures in living animals. Importantly, there is a high level of genetic conservation between C. elegans and humans: ˜50% of human genes have a C. elegans homolog including 81% of human kinases. Moreover, major signaling pathways such as RTK-Ras-MAPK, Insulin/IGR, TOR, Notch, Wnt, TGF-β, and G-Protein Coupled Receptors are conserved. Thus, the C. elegans strains are an attractive model for (1) screening small molecules affecting conserved pathways, (2) identifying drug targets, (3) hit prioritization and allowing for “fast failing” compounds and (4) discovering new disease-causing genes.

Provided herein are tools and methods for screening, e.g., a HTS platform for automated genome-wide screening of RNA interference (RNAi)-mediated gene inactivations, or in some embodiments, chemically-generated worm mutants. Gene inactivations can be analyzed for elimination/resistance or enhancement of the hit's effect. In these or other embodiments, directed gene editing is conducted on gene targets using, for example, CRISPR/CAS9 technology to probe candidate genes and pathways identified for target confirmation and for development of tools for further biochemical or genetic analyses.

In some aspects, the invention provides Caenorhabditis elegans (C. elegans) strains exhibiting an RNA toxicity phenotype. The C. elegans strain comprises a detectable reporter gene expressed in one or more cell types, with the expressed reporter gene RNA having pathogenic or non-pathogenic oligonucleotide repeats. The C. elegans strains described herein are useful for high-throughput screening applications, for identification of gene targets involved in RNA toxicity disorders, as well as for small molecule identification for therapeutic agents that ameliorate RNA toxicity disorders, e.g., DM1.

In some embodiments, the reporter gene RNA has at least about 70 repeats of the oligonucleotide (e.g., trinucleotide), so as to display an RNA toxicity phenotype. In some embodiments, the strain contains a reporter gene having at least about 100 repeats of the oligonucleotide, or at least about 120 repeats of the oligonucleotide, or at least about 150 repeats of the oligonucleotide, or at least about 175 repeats of the oligonucleotide, or at least about 200 repeats of the oligonucleotide, or at least about 225 repeats of the oligonucleotide, or at least about 250 repeats of the oligonucleotide, or at least about 500 repeats of the oligonucleotide. In some embodiments, the reporter gene has up to about 500, 1000, 1500, 2000, 2500, or 5000 repeats of the oligonucleotide. These pathogenic levels of oligonucleotide repeat exhibit a length-dependent decline in adult stage reporter gene protein levels. Further, by visualizing cellular localization of the RNA, the reporter gene RNA is seen to accumulate into nuclear foci. These phenotypes allow for highly effective high-throughput screening (HTS) systems, to identify gene pathways and targets involved in RNA toxicity, and also for identification of therapeutic agents that may ameliorate RNA toxicity. The C. elegans strain in some embodiments will also display a motor defect in the adult stage, providing additional functional assays to elucidate the biology of RNA toxicity disorders, as well as the identification of therapeutic agents that may ameliorate RNA toxicity.

As used herein, the term “about” means±10% of the associated numerical value.

The invention further provides control C. elegans strains, which also have detectable reporter genes with oligonucleotide repeat regions, but at a non-pathogenic level, such as less than about 50, or less than about 40 oligonucleotide repeats (e.g., trinucleotide repeats). In some embodiments, the control strain has a detectable reporter gene having a region of at least 10, but less than about 50 trinucleotide repeats. These control strains are also useful for HTS systems, to provide control levels of the detectable reporter protein, non-pathogenic animal motility, as well as non-pathogenic cellular localization of the reporter gene RNA.

The detectable reporter gene can be expressed from the C. elegans chromosome, or can be expressed extrachromosomally. In some embodiments, the detectable reporter gene is stably integrated into the C. elegans genome. Methods are well known for integrating exogenous DNA into the C. elegans genome. Generally, extrachromosomal arrays are integrated into a chromosome to reduce their genetic instability and variability. Methods for integrating arrays include irradiation of transgenic strains, which presumably induces chromosomal breaks and ligation of arrays to chromosomes during DNA repair. Because of this, mutations can arise, so it is preferable to outcross the recovered integrated strains by mating with wild type worms. Alternatively, transgene DNAs can be co-injected with a single stranded DNA oligonucleotide. The oligonucleotide may stimulate random integration and/or suppresses array formation.

In some embodiments, the reporter gene is expressed from a tissue-specific promoter. Expression in different tissues can aid in identification of different genes potentially involved in RNA toxicity. In some embodiments, the reporter gene is expressed in body wall muscle cells. In some embodiments, the reporter gene is expressed in neurons. C. elegans has been extensively characterized, and lists of cell-type and location specific promoters are known in the art (see, for example, C. elegans II, second edition, Cold Spring Harbor Monograph Series, Vol 33, Cold Spring Harbor Press, Cold Spring Harbor, N.Y. (1997), and wormbase.org. For example, neuron-specific promoters include, ace-1, acr-5, aex-3, apl-1, alt-1, cat-1, cat-2, cch-1, cdh-3, ceh-2, ceh-2, ceh-6, ceh-10, ceh-14, ceh-17, ceh-23, ceh-28, ceh-36, che-1, che-3, cfi-1, cgk-1, cha-1, cnd-1, cod-5, daf-1, daf-4, daf-7, daf-19, dbl-1, des-2, deg-1, deg-3, del-1, eat-4, eat-16, ehs-1, egl-10, egl-17, egl-19, eg1-2, eg1-36, eg1-5, eg1-8, fax-1, flp-1, flp-1, flp-3, flp-5, flp-6, flp-8, flp-12, flp-13, flp-15, flp-3, fir-4, gcy-10, gcy-12, gcy-32, gcy-33, gcy-5, gcy-6, gcy-7, gcy-8, ggr-1, ggr-2, ggr-3, glr-1, glr-5, glr-7, glt-1, goa-1, gpa-1, gpa-1, gpa-2, gpa-3, gpa-4, gpa-5, gpa-6, gpa-7, gpa-8, gpa-9, gpa-10, gpa-11, gpa-13, gpa-14, gpa-15, gpa-16, gpb-2, gsa-1, ham-2, her-1, ida-1, lim-4, lim-6, lim-6, lim-7, lin-11, lin-4, lin-45, mab-18, mec-3, mec-4, mec-7, mec-8, mec-9, mec-18, mgl-1, mgl-2, mig-1, mig-13, mus-1, ncs-1, nhr-22, nhr-38, nhr-79, nmr-1, ocr-1, ocr-2, odr-1, odr-2 odr-10, odr-3, odr-3, odr-7, opt-3, osm-10, osm-3, osm-9, pag-3, pef-1, pha-1, pin-2, rab-3, ric-19, sak-1, sdf-13, sek-1, sek-2, sgs-1, snb-1, snt-1, sra-1, sra-10, sra-11, sra-6, sra-7, sra-9, srb-6, srg-2, srg-1, srd-1, sre-1, srg-13, sro-1, str-1, str-2, str-3, syn-2, tab-1, tax-2, tax-4, tig-2, tph-1, ttx-3, ttx-3, unc-3, unc-4, unc-5, unc-8, unc-11, unc-17, unc-18, unc-25, unc-29, unc-30, unc-37, unc-40, unc-3, unc-47, unc-55, unc-64, unc-86, unc-97, unc-103, unc-115, unc-116, unc-119, unc-129, and vab-7 promoters. Muscle-specific promoters include the hlh-1, mlc-2, myo-3, unc-54 and unc-89 promoters. In some embodiments, the detectable reporter gene is expressed under control of the myo-3 promoter. Expression of the detectable reporter gene can also be targeted to other cell types, such as the pharynx (pharynx specific promoters include the ceh-22, hlh-6 and myo-2 promoters); and gut (gut-specific promoters include the nhx-2, vit-2, cpr-1, ges-1, mtl-1, mtl-2, pho-1, spl-1, vha-6 and elo-6 promoters).

In some embodiments, the detectable reporter gene encodes a fluorescent or luminescent protein. Various fluorescent proteins that fluoresce in vivo are known in the art, including, but not limited to, green fluorescent protein, enhanced green fluorescent protein, red fluorescent protein, yellow fluorescent protein, etc. For example, in some embodiments, the detectable reporter gene encodes a green fluorescent protein (GFP). In other embodiments, the detectable reporter gene is selected from luciferase, a modified luciferase protein, blue/UV fluorescent proteins (for example, TagBFP, Azurite, EBFP2, mKalama1, Sirius, Sapphire, and T-Sapphire), cyan fluorescent proteins (for example, ECFP, Cerulean, SCFP3A, mTurquoise, monomeric Midoriishi-Cyan, TagCFP, and mTFP1), green fluorescent proteins (for example, EGFP, Emerald, Superfolder GFP, Monomeric Azami Green, TagGFP2, mUKG, and mWasabi), yellow fluorescent proteins (for example, EYFP, Citrine, Venus, SYFP2, and TagYFP), orange fluorescent proteins (for example, Monomeric Kusabira-Orange, mKOK, mKO2, mOrange, and mOrange2), red fluorescent proteins (for example, mRaspberry, mCherry, mStrawberry, mTangerine, tdTomato, TagRFP, TagRFP-T, mApple, and mRuby), far-red fluorescent proteins (for example, mPlum, HcRed-Tandem, mKate2, mNeptune, and NirFP), near-IR fluorescent proteins (for example, TagRFP657, IFP1.4, and iRFP), long stokes-shift proteins (for example, mKeima Red, LSS-mKate1, and LSS-mKate2), photoactivatible fluorescent proteins (for example, PA-GFP, PAmCherryl, and PATagRFP), photoconvertible fluorescent proteins (for example, Kaede (green), Kaede (red), KikGR1 (green), KikGR1 (red), PS-CFP2, PS-CFP2, mEos2 (green), mEos2 (red), PSmOrange, and PSmOrange), and photoswitchable fluorescent proteins (for example, Dronpa).

In some embodiments, the oligonucleotide repeats are in the 3′ and/or 5′ UTR of the detectable reporter gene, or in an intron, or in other embodiments the oligonucleotide repeat is in a coding region. The oligonucleotide repeats are generally repeats of from 3 to 6 nucleotides, and in some embodiments are trinucleotide repeats. In some embodiments, the trinucleotide repeat is selected from CUG, CAG, CGG, CCG, GAA, or CTG, and can be selected to mimic the trinucleotide repeat in a corresponding human condition. In some embodiments, the strain mimics a polyglutamine disorder, where the trinucleotide encodes glutamine, and the repeat is in a coding region. In other embodiments, the strain mimics a non-polyglutamine disorder, and the trinucleotide repeat is in a non-coding region.

In some embodiments, the trinucleotide repeat regions can mimic conditions such as DM1, in which expansions in a CUG repeat in the 3′ UTR of a protein kinase gene leads to the RNA toxicity phenotype. In other embodiments, the C. elegans strain mimics the trinucleotide repeats found in Fragile X syndrome (CGG) or spinocerebellar ataxia (e.g., types 2, 8, 10, and 12). In other embodiments, the trinucleotide repeats may encode polyglutamine (e.g., CAG repeats). Where the trinucleotide repeats are in the coding region they can mimic pathologies observed in conditions such as Huntington's disease-like 2 (polyglutamine condition). Thus, in some embodiments, the trinucleotide repeats are CUG repeats, and are in the non-coding regions, such as the 3′ UTR. In some embodiments, the trinucleotide repeats are CGG or CAG repeats, and may be in coding or non-coding regions. In still other embodiments, besides occurring in distinct localizations, RNA-associated repeats can be tetranucleotides, such as CCTG expanded repeats as observed in in Myotonic Dystrophy 2 (DM2), or hexanucleotides, such as GGGGCC expanded repeats observed in Amyotrophic Lateral Sclerosis.

The RNA toxicity phenotype of these strains allows for the biology of the condition to be explored through a series of gene inactivations, mutations, or overexpressions, which can be screened for impact on the pathology in high throughput in some embodiments. Thus, in this aspect, genes are identified with the potential to ameliorate or enhance the pathologic phenotype. In some embodiments, the C. elegans strain further comprises an inactivation or overexpression of at least one endogenous gene. For example, the C. elegans strain may comprise a modification or inactivation of at least one endogenous gene, which can be created by any mutagenesis or gene expression modification technique, including RNAi or gene editing technology (e.g., CRISPR/CAS9).

In some embodiments, the endogenous gene encodes a signaling protein (e.g., a kinase, a phosphatase, or a GPCR), a protein involved in RNA processing or degradation (including nonsense-mediated mRNA decay pathways), RNA transport, transcription, DNA repair or recombination, or translation. In some embodiments, the endogenous gene encodes a protein of the nonsense-mediated mRNA decay (NMD) pathway. In some embodiments, the endogenous gene is a gene listed in Table 2 or Table 3. For example, the endogenous gene may be str-67, ocrl-1, an ortholog of human KRTAP5-7, an ortholog of human ADCY4, nol-9, smg-2, npp-4, asd-1, dpy-22, hda-2, mrt-2, grid-1, ortholog of human CSTF2T, cfim-2, or ortholog of human DIS3L2.

Also described herein are multiwell plates that have a C. elegans strain as described herein in each of a plurality of wells (e.g., all of the wells may have the same strain, or a plurality of different strains, e.g., with each different strain in one, two, or more wells). One or more wells may further contain a C. elegans strain that does not exhibit an RNA toxicity phenotype. In various embodiments, the multiwell plate may comprise from ten to twenty C. elegans organisms per well. The multiwell plates may contain C. elegans in at least 50, at least 75, or at least 100, or at least 200, or at least 300, or at least 500, or at least 1000 wells, allowing high-throughput screening. The multiwell plate may contain a C. elegans strain in accordance with the invention, each having a different gene inactivation, overexpression, or modification, for screening effects on the pathogenic phenotype. In some embodiments, the multiwell plate provides strains with inactivations, overexpressions, or modifications in endogenous genes encoding signaling proteins (e.g., a kinase, a phosphatase, or a GPCR), proteins involved in RNA processing or degradation (including nonsense-mediated mRNA decay pathways), RNA transport, transcription, DNA repair or recombination, and/or translation. In some embodiments, the multiwell plate screens inactivations or modifications or overexpressions in a plurality (e.g., at least 2, at least 5, or at least 10) endogenous gene encoding proteins of the nonsense-mediated mRNA decay (NMD) pathway. In some embodiments, the C. elegans contain inactivations, overexpressions, or modifications of genes listed in Table 2 and/or Table 3.

In some aspects, the invention provides a method for identifying an agent that modulates an RNA toxicity phenotype. The methods can comprise providing the multiwell plate described above, and adding a candidate agent to each of a plurality of wells, and quantifying an effect on said RNA toxicity phenotype. In these embodiments, the C. elegans need not contain any inactivations, overexpression, or modifications of any endogenous genes, that is, all experimental (i.e., non-control) wells contain the identical C. elegans strain. Control wells containing C. elegans strains that do not exhibit the RNA toxicity phenotype, or exhibit a reduced toxicity phenotype, are typically included. Although methods using multiwall plates are exemplified, other formats may also be used, e.g., low- or medium-throughput or other formats.

In some embodiments, the effect on said RNA toxicity phenotype is quantified by the level of protein expression of said reporter gene and/or cellular location of the reporter gene RNA. In some embodiments, the effect on said RNA toxicity phenotype is quantified by the accumulation of RNA into nuclear foci. Level of reporter protein expression is easily quantified in high throughput by simple measurement of, for example, protein fluorescence. Cellular location and accumulation of RNA into nuclear foci can be detected and quantified by in situ hybridization techniques, including FISH. Signals can be quantified in high throughput in some embodiments by imaging the wells and measuring intensity, e.g., pixel-by-pixel, of the images. Cellular components, such as the nucleus, can be visualized in parallel in some embodiments using known techniques, such a DAPI stain.

In these or other embodiments, the method may comprise quantifying a change in motility. For example, in some embodiments, worms showing reduced or enhanced toxicity phenotypes by reporter protein expression and/or RNA accumulation in the nucleus are further evaluated for motility defects. Without limitation, motility can be evaluated and quantified by measuring the percentage of animals that reach a food attractant, the velocity of animals toward a food attractant, or general improvement in animal motility without attractant. Motility, including velocity or general movement, may be evaluated or measured in solid or liquid.

In various embodiments, after high throughput screening of candidate therapeutic agents, an agent is selected that reduces the RNA toxicity phenotype, either by one or more (e.g., all) of increasing reporter protein expression, reducing accumulation of RNA in the nucleus, or reducing motility defects. Effective agents can be selected and tested in further animal models, including mammalian models of RNA toxicity disease, and/or used to dose human patients.

In another aspect, the invention provides a method for making a pharmaceutical composition for treatment of a condition associated with RNA toxicity. In these embodiments, the method comprises identifying an agent that reduces RNA toxicity phenotype using the C elegan strains, multiwell plate formats, and/or assays described above, and formulating said agent as a pharmaceutically acceptable composition. For example, the agent may be formulated for systemic administration, including in conventional oral formulations such as tablets, capsules, or pills, or formulated for parenteral administration, including for intravenous, subcutaneous, or intramuscular injection, s described further below.

In various embodiments, the candidate agents and therapeutic agents are small molecule, nucleic acid, polypeptide, or peptide compounds, or analogues thereof. The agent can be any chemical entity, including, without limitation, synthetic and naturally-occurring proteinaceous and non-proteinaceous entities. In some embodiments, the agent is a nucleic acid, a nucleic acid analogue, a protein, an antibody, a peptide or peptide analogue, an aptamer, an oligomer of nucleic acids, an amino acid or amino acid analogue, or a carbohydrate, and includes, without limitation, proteins, oligonucleotides, ribozymes, DNAzymes, glycoproteins, antisense oligonucleotides, siRNAs, lipoproteins, aptamers, and modifications and combinations thereof etc.

In some embodiments, the therapeutic agent is a small molecule. As used herein, the term “small molecule” refers to a chemical agent that is an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

In various embodiments, said agent inhibits the expression or activity of a gene selected from Table 2 or 3. In some embodiments, said agent increases the expression or activity of a gene selected from Table 2 or 3. In some embodiments, the gene is involved in the nonsense-mediated mRNA decay pathway, or is a signaling protein. For example, the agent may inhibit the expression or activity of one or more of str-67, ocrl-1, or an ortholog of human KRTAP5-7, and in some embodiments, inhibits the expression or activity of a human ortholog. In some embodiments, the agent increases the expression or activity or one or more of an ortholog of ADCY4, nol-9, smg-2, npp-4, asd-1, dpy-22, hda-2, mrt-2, grld-1, ortholog of human CSTF2T, cfim-2, or ortholog of human DIS3L2, and in some embodiments, the agents increases the expression or activity of a human ortholog.

In various embodiments, the present invention provides for preparation of pharmaceutical compositions comprising the agent, and a pharmaceutically acceptable carrier or excipient. Exemplary excipients include sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpolypyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients may have two or more functions in the oral dosage form.

Pharmaceutical compositions may be administered to patients by any route which is compatible with the particular compound or pharmaceutically composition. It is contemplated that the compositions be provided to a subject by any suitable means, directly (e.g., locally, as by injection, implantation or topical administration to a tissue) or systemically (e.g., parenterally or orally). In an embodiment, the pharmaceutical composition is administered orally. In another embodiment, the pharmaceutical composition is administered parenterally. In an embodiment, the pharmaceutical composition is administered by intravenous or subcutaneous injection.

The pharmaceutical composition can take the form of solutions, suspensions, emulsion, drops, tablets, pills, pellets, capsules, capsules containing liquids, gelatin capsules, powders, suppositories, emulsions, aerosols, sprays, suspensions, lyophilized powder, frozen suspension, dessicated powder, delayed-release formulations, sustained-release formulations, controlled-release compositions, nanoparticle formulations, or any other form suitable for use.

Pharmaceutical compositions for parenteral delivery may contain, for example, suspending or dispersing agents known in the art. Exemplary suspending agents include, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof. Additional components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

The formulations comprising the therapeutic agents may be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In still other aspects, the invention provides a method for treating a condition characterized by RNA toxicity. In these embodiments, the method comprises administering the pharmaceutical composition prepared according to the method described above to a patient in need. In some embodiments, the patient has myotonic dystrophy 1 (DM1). In other embodiments, the patient has Fragile X syndrome, Huntington's disease-like 2, spinocerebellar ataxia, or amyotrophic lateral sclerosis, or other disorder characterized by RNA toxicity resulting from oligonucleotide repeat expansion, including trinucleotide repeat expansion.

It will be appreciated that the actual dose of the therapeutic agent to be administered according to the present invention will vary according to the particular compound, the particular dosage form, the mode of administration, and the particular disorder and condition of the patient. Many factors that may modify the action of the therapeutic agent (e.g., body weight, gender, diet, time of administration, route of administration, rate of excretion, condition of the subject, drug combinations, genetic disposition and reaction sensitivities) can be taken into account by those skilled in the art.

The desired dose of the therapeutic agent may be presented as one dose or two or more sub-doses administered at appropriate intervals throughout the dosing period. In accordance with certain embodiments of the invention, the pharmaceutical composition is administered, more than once daily, about once per day, about every other day, about every third day, about once a week, about once every two weeks, about once every month. In an embodiment, the pharmaceutical composition is administered more than once daily, for example, twice, three times, four times, five times, or six times daily. In another embodiment, the pharmaceutical composition is administered once daily. In some embodiments, the regimen is continued for at least one month, at least six months, at least nine months, or at least one year. In various embodiments, the pharmaceutical composition is administered from 1 to 3 times daily to ameliorate symptoms of the disease.

### EXAMPLES

The invention is further described in the following examples, which do not limit the scope of the invention described in the claims.

### Example 1

### Identification of Genes in Trinucleotide Repeat RNA Toxicity Pathways in C. elegans

Myotonic dystrophy disorders are caused by expanded CUG repeats in non-coding regions. To reveal mechanisms of CUG repeat pathogenesis we used C. elegans expressing CUG repeats to identify gene inactivations that modulate CUG repeat toxicity.

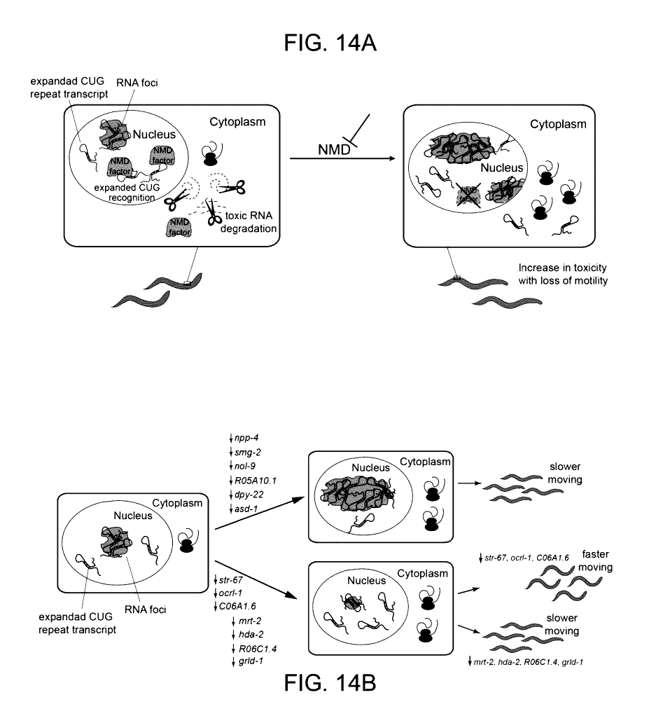

The gene inactivations that modulate phenotypes of expanded CUG RNA repeats comprise multiple pathways beyond splicing dysregulation. Demonstrated herein are a number of previously unknown genes that are involved as modulators of expanded CUG toxicity and expanded CUG repeat foci formation. The demonstration that different gene inactivations, all expanded CUG repeat toxicity suppressors, have opposing effects on foci accumulation (Table 3, FIG. 14B), supports the hypothesis that these genes act in distinct pathways. Genes where a direct correlation exists between expanded CUG repeat toxicity and foci accumulation (FIG. 14B) include genes where modulation of expanded RNA toxicity can occur by: clearance of CUG-containing RNA transcripts, binding of expanded CUG RNA preventing foci formation or promotion of mRNA transport from the nucleus. Inactivation of these genes causes an increase in the toxic expanded CUG species present in the nucleus. One example is smg-2/NMD helicase inactivation. Another class of suppressor gene inactivations does not correlate with an increase in foci formation (FIG. 14B); these proteins may detect cellular damage or bind to expanded CUG repeats.

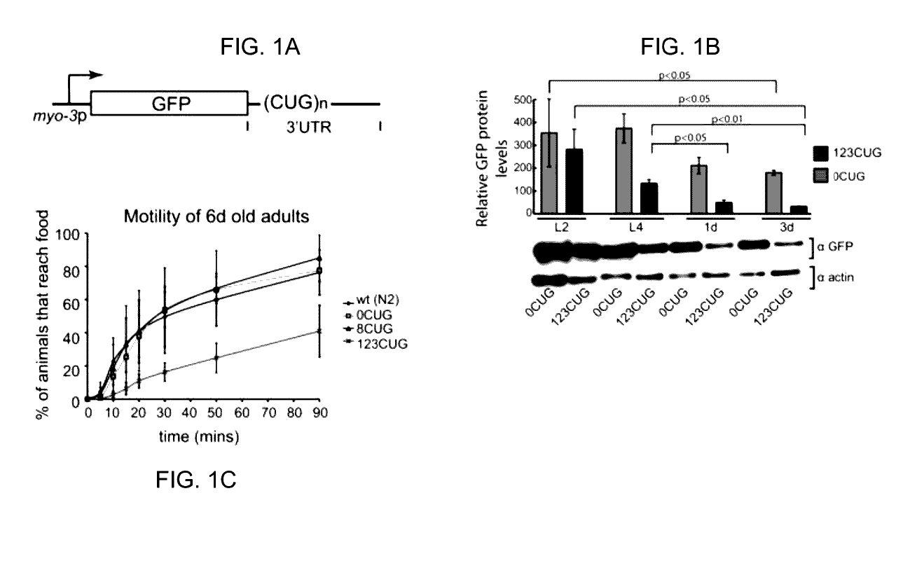

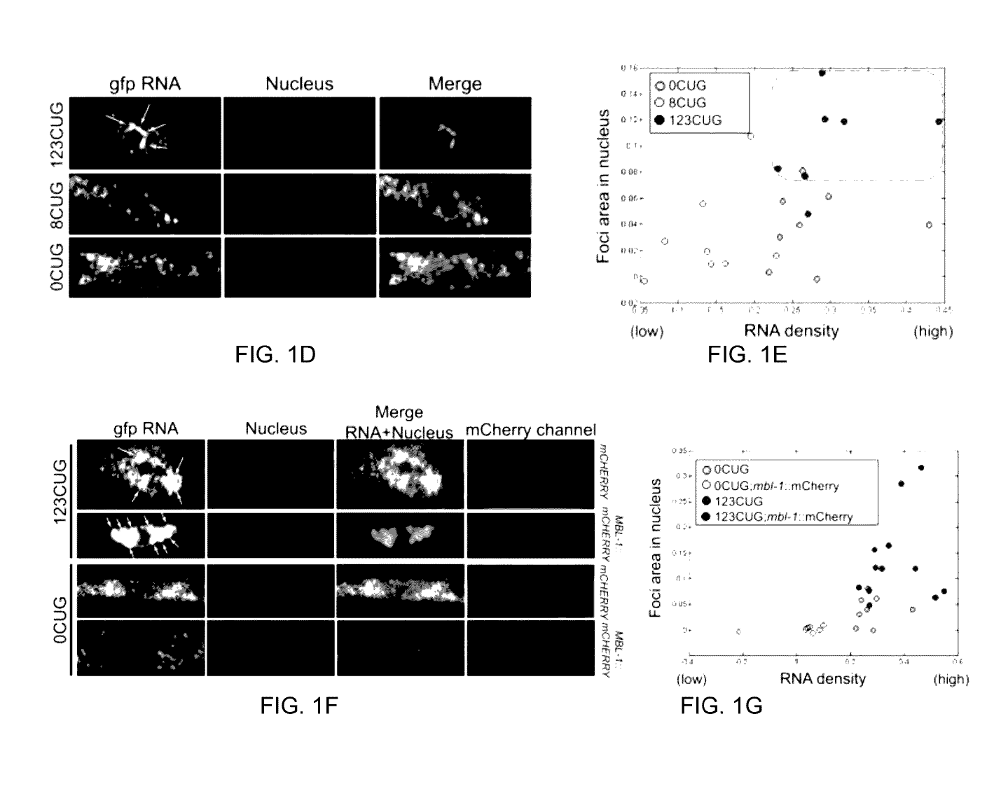

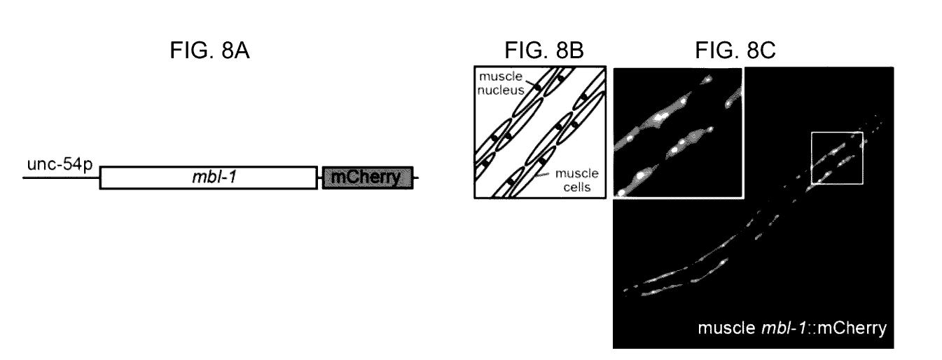

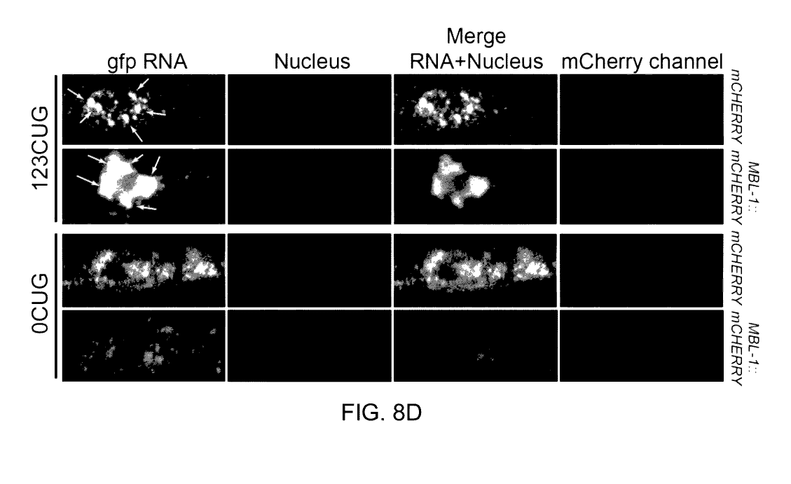

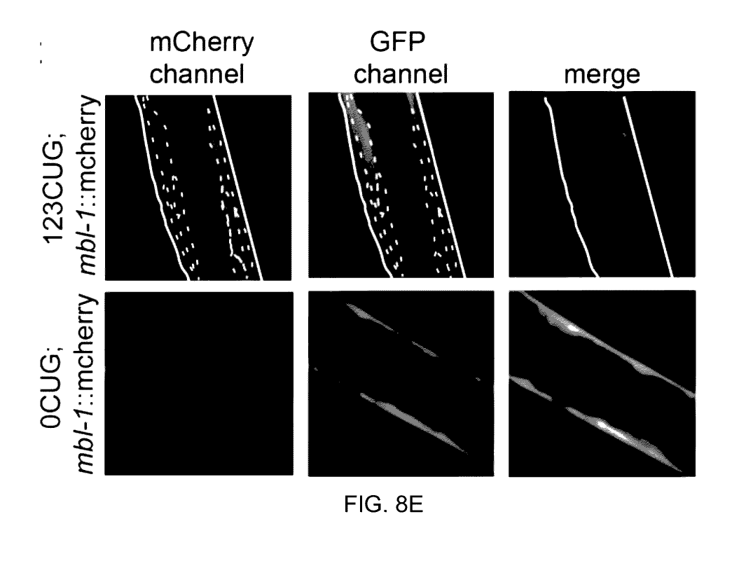

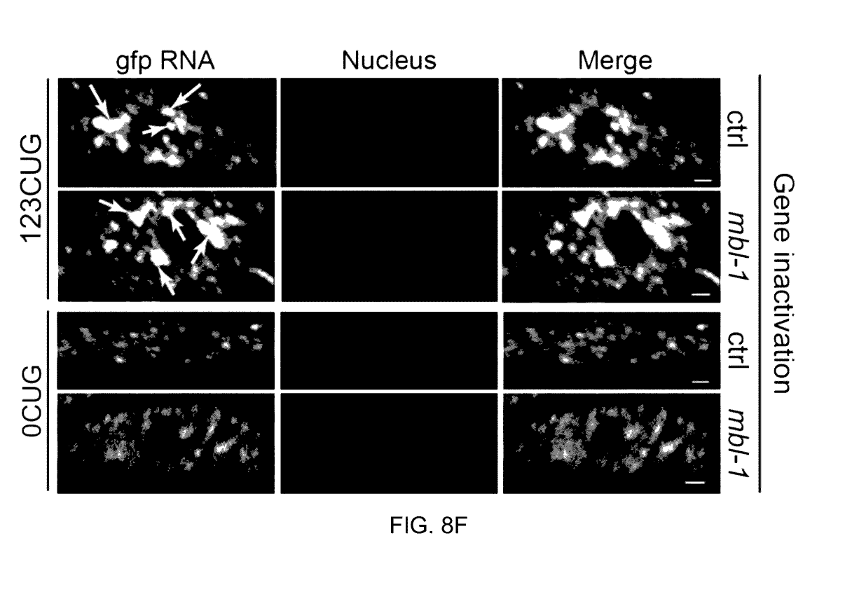

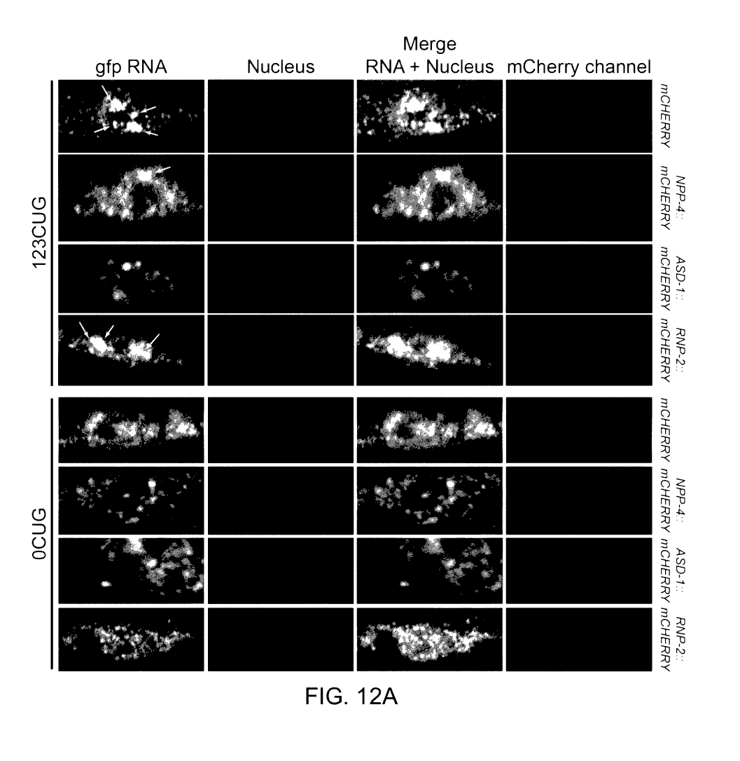

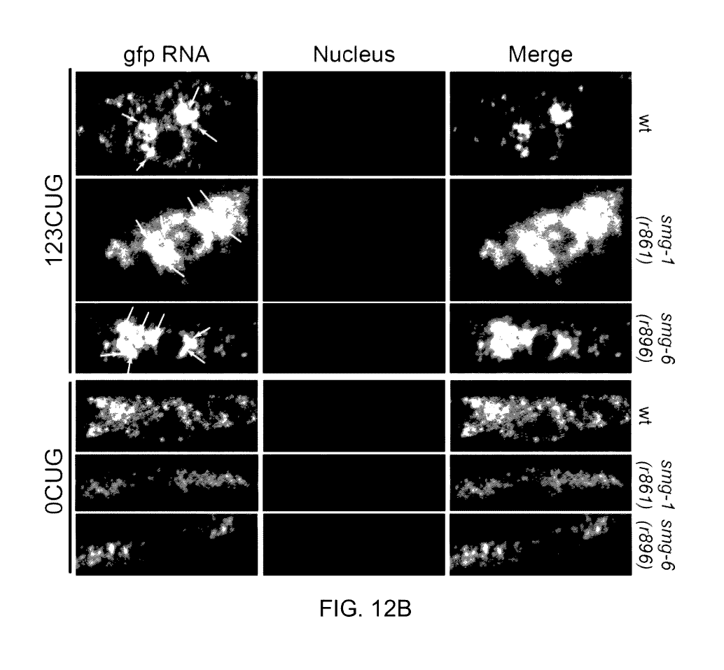

The identification in the screen of additional splicing factors, such as the asd-1 and grld-1 genes, that when inactivated caused an increase in expanded CUG toxicity, was reasonable (Table 3, below). Unlike MBL1 overexpression (FIG. 1F, FIG. 8D), ASD-1 overexpression led to a decrease in expanded CUG nuclear foci accumulation (FIG. 12). ASD-1 is an alternative splicing factor and belongs to the Fox-1 splicing family. In vertebrates, MBNL genes are silenced by Fox-1/2 splicing factors. Two mechanisms for ASD-1 suppression of expanded CUG repeat toxicity emerge: 1) ASD-1 regulates functional MBNL1 levels available by modulating splicing variants; 2) ASD-1 may bind directly or indirectly to expanded CUG repeats and affect toxicity.

Most of the gene inactivations identified make the response to expanded CUG repeats more toxic and promote the accumulation of larger RNA foci in the nuclei, suggesting that these genes constitute a CUG repeat detoxification pathway that blunts their toxicity.

Commonalities have been suggested in degenerative pathways between repeat-based RNA-mediated disorders, and protein-mediated disorders. RNA toxicity has been implicated in polyQ expansion disorders, and MBNL1 functions as a modulator of polyQ toxicity through its interaction with CAG-containing RNA transcripts. A subset of the genes identified in the screen as modifiers of expanded CUG toxicity are modulators of polyQ aggregation or toxicity, hda-2, mrt-2 and smg-2 genes. npp-4, although not previously linked to repeat expansion disorders, is part of the nuclear pore complex together with npp-8, and npp-8 had been identified as a modulator of polyQ aggregation. The identification of pathways that function as common regulators to a broad class of triplet nucleotide pathogenic expansions supports the model of common toxic mechanisms for coding and non-coding triplet repeat disorders.

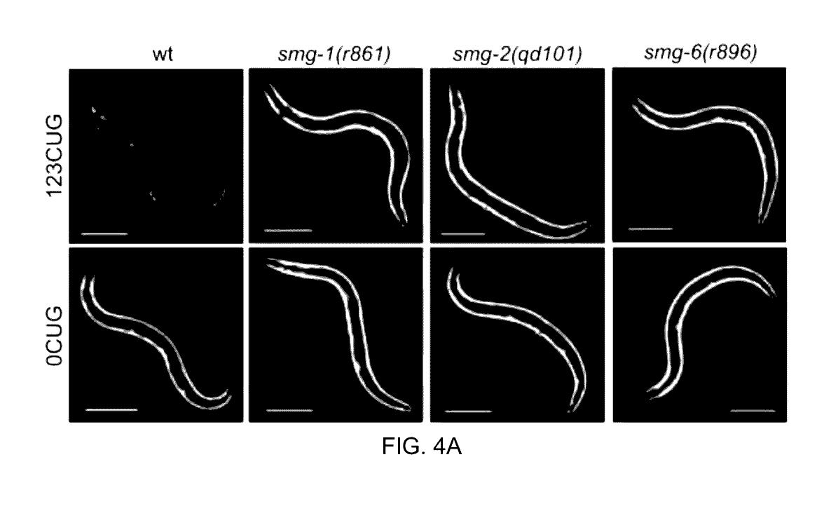

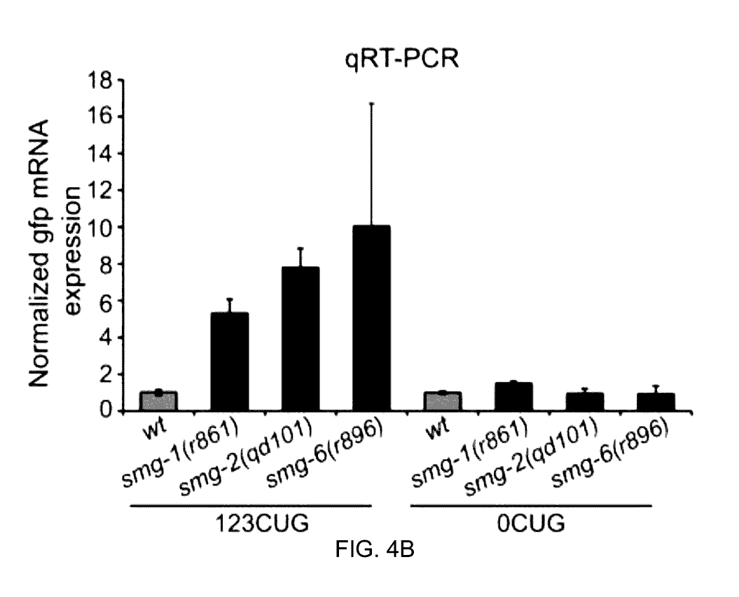

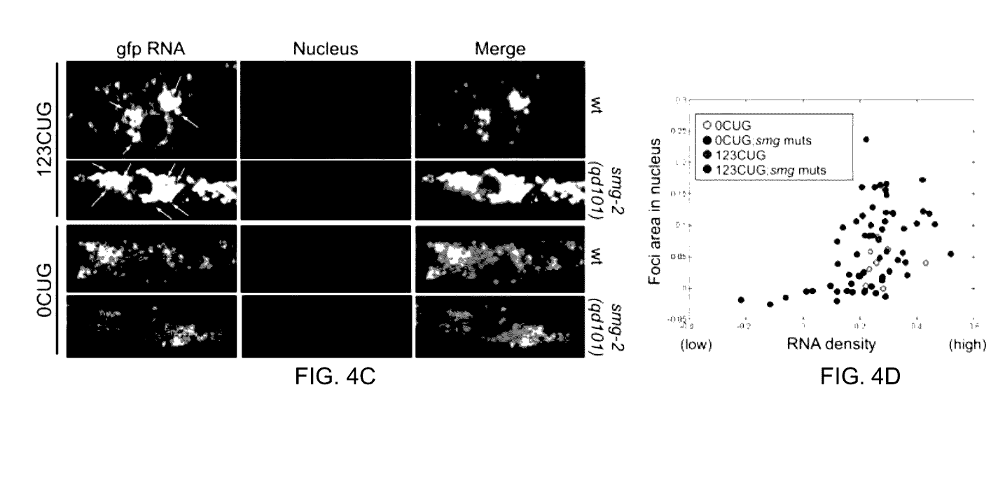

The NMD pathway is a conserved mechanism of mRNA surveillance that regulates the expression of 5-10% of the human, D. melanogaster and yeast transcriptomes. In addition to its expected target transcripts, NMD modulates the abundance of transcripts containing CUG repeats in their 3′UTR, reducing the accumulation and nuclear foci formation of these toxic RNA species (FIG. 14) in both C. elegans and human cells (FIGS. 4 and 6 and FIG. 12B). Sequence composition is key in the recognition by NMD of RNA transcripts containing 3′UTR CUGs; a similar G/C-rich (≈66%) sequence, when present in the 3′UTR, is also recognized by NMD, whereas an A/T-rich sequence is not.

With the identification of NMD genes as modulators of expanded CAG repeat protein-based disorders, these results suggest broader surveillance roles for the NMD pathway. RNA transcripts containing expanded CAG repeats, also GC-rich, are likely to form secondary structures that may directly or indirectly trigger the NMD pathway. Additionally, NMD has been mapped to nuclear surveillance leading to nuclear RNA degradation as well as cytoplasmic degradation. These data showing a striking accumulation of nuclear RNA foci and cytoplasmic RNA foci in NMD mutants suggests a role for NMD not only in the cytoplasm but also in nuclear clearance of expanded RNA repeat transcripts.

Modulation of the NMD pathway may offer a therapeutic approach for myotonic dystrophy patients as well as other repeat-based degenerative disorders. Pharmacological compounds that increase NMD pathway activity may clear CUG-containing RNA toxic species, with the potential to significantly ameliorate DM-related symptoms. NMD efficiency varies across tissues and between individuals, with significant clinical implications. These variations in NMD efficiency may have significant implications for trinucleotide repeat disease onset or progression.

### Methodology.

The following materials and methods were used in Example 1.

### Plasmids and Constructs

Mammalian CTG repeat sequences were amplified from plasmids pR26eGFP+100 and pR26eGFP+20039 using Extended High Fidelity from Roche in 6% DMSO and 1M betaine (Sigma). CTG repeats were cloned into the C. elegans pPD118.20 vector bearing the myo-3 body wall muscle-specific promoter, GFP, and the let-858 3′ UTR. The mbl-1 and rnp-2 genes were amplified from C. elegans N2 genomic DNA, and asd-1 and npp-4 from cDNA, using Phusion polymerase (Finnzymes). These genes were cloned into the C. elegans vectors pPD49.26 and pPD30.38 (Addgene) bearing the unc-54 body wall muscle-specific promoter. The GC-rich and AT-rich nucleotide sequences were cloned from the coding region of the 1,4-alpha-glucan branching enzyme gene of Pseudomonas aeruginosa (glgB) and the 3′utr region of the Arabidopsis thaliana myb domain protein 51 gene (myb51), respectively. The synthetic GC-rich and AT rich sequences were synthesized (GenScript). The GC-rich and AT-rich sequences were amplified and cloned into the C. elegans pPD118.20 (Addgene) vector bearing the myo-3 body wall muscle-specific promoter.

C. elegans Strains

Nematodes were handled using standard methods and experiments were performed at 20° C., unless otherwise indicated. The C. elegans N2 Bristol strain was used as wild-type strain. Strains generated for this study are indicated in Table 1.

Transgenes containing gfp fused to different CTG lengths were integrated by exposing animals to UV irradiation and strains were outcrossed 5 times. Several independent strains were obtained carrying the different GFP transgenes and the different strains generated exhibited similar length-dependent phenotypes. The remaining transgenic strains expressed their transgenes as extrachromosomal arrays.

TABLE 1

| Strain | Genotype |
|---|---|
| GR2024 | mgIs64[myo-3p::gfp::3′utr123(CUG)] |
| GR2025 | mgIs65[myo-3p::gfp::3′utr0(CUG)] |
| GR2026 | mgIs66[myo-3p::gfp::3′utr8(CUG)] |
| GR2027 | mgEx780[unc-54p::mbl-1::mcherry] |
| GR2028 | mgEx781[unc-54p::mcherry] |
| GR2029 | mgEx782[unc-54p::npp-4::mcherry] |
| GR2030 | mgEx783[unc-54p::asd-1::mcherry] |
| GR2031 | mgEx784[unc-54p::rnp-2::mcherry] |
| GR2032 | mgIs64[myo-3p::gfp::3′utr123(CUG)]; |
| | mgEx780[unc-54p::mbl-1::mcherry] |
| GR2033 | mgIs65[myo-3p::gfp::3′utr0(CUG)]; |
| | mgEx780[unc-54p::mbl-1::mcherry] |
| GR2034 | mgIs64[myo-3p::gfp::3′utr123(CUG)]; |
| | mgEx781[unc-54p::mcherry] |
| GR2035 | mgIs65[myo-3p::gfp::3′utr0(CUG)]; |
| | mgEx781[unc-54p::mcherry] |
| GR2036 | mgIs64[myo-3p::gfp::3′utr123(CUG)]; |
| | mgEx782[unc-54p::npp-4::mcherry] |
| GR2037 | mgIs65[myo-3p::gfp::3′utr0(CUG)]; |
| | mgEx782[unc-54p::npp-4::mcherry] |
| GR2038 | mgIs64[myo-3p::gfp::3′utr123(CUG)]; |
| | mgEx783[unc-54p::asd-1::mcherry] |
| GR2039 | mgIs65[myo-3p::gfp::3′utr0(CUG)]; |
| | mgEx783[unc-54p::asd-1::mcherry] |
| GR2040 | mgIs64[myo-3p::gfp::3′utr123(CUG)]; |
| | mgEx784[unc-54p::rnp-2::mcherry] |
| GR2041 | mgIs65[myo-3p::gfp::3′utr0(CUG)]; |
| | mgEx784[unc-54p::rnp-2::mcherry] |
| GR2042 | mgEx785[myo-3p::gfp::3′utr(GC-rich_long)] |
| GR2043 | mgEx786[myo-3p::gfp::3′utr(AT-rich_long)] |
| GR2077 | mgIs64[myo-3p::gfp::3′utr123(CUG)]; smg-1(r861) |
| GR2078 | mgIs64[myo-3p::gfp::3′utr123(CUG)]; smg-2(qd101) |
| GR2079 | mgIs64[myo-3p::gfp::3′utr123(CUG)]; smg-6(r896) |
| GR2080 | mgIs65[myo-3p::gfp::3′utr0(CUG)]; smg-1(r861) |
| GR2081 | mgIs65[myo-3p::gfp::3′utr0(CUG)]; smg-2(qd101) |
| GR2082 | mgIs65[myo-3p::gfp::3′utr0(CUG)]; smg-6(r896) |
| GR2083 | mgEx787[myo-3p::gfp::3′utr(GC-rich_short)] |
| GR2084 | mgEx787[myo-3p::gfp::3′utr(GC-rich_short)]; smg-1(r861) |
| GR2085 | mgEx787[myo-3p::gfp::3′utr(GC-rich_short)]; smg-2(qd101) |
| GR2086 | mgEx787[myo-3p::gfp::3′utr(GC-rich_short)]; smg-6(r896) |
| GR2087 | mgEx788[myo-3p::gfp::3′utr(AT-rich_short)] |
| GR2088 | mgEx788[myo-3p::gfp::3′utr(AT-rich_short)]; smg-1(r861) |
| GR2089 | mgEx788[myo-3p::gfp::3′utr(AT-rich_short)]; smg-2(qd101) |
| GR2090 | mgEx788[myo-3p::gfp::3′utr(AT-rich_short)]; smg-6(r896) |
| GR2091 | mgEx789[myo-3p::gfp::3′utr(31% GCinsert)] |
| GR2092 | mgEx789[myo-3p::gfp::3′utr(31% GCinsert)]; smg-1(r861) |
| GR2093 | mgEx789[myo-3p::gfp::3′utr(31% GCinsert)]; smg-2(qd101) |
| GR2094 | mgEx789[myo-3p::gfp::3′utr(31% GCinsert)]; smg-6(r896) |
| GR2095 | mgEx790[myo-3p::gfp::3′utr(32% GCinsert)] |
| GR2096 | mgEx790[myo-3p::gfp::3′utr(32% GCinsert)]; smg-1(r861) |
| GR2097 | mgEx790[myo-3p::gfp::3′utr(32% GCinsert)]; smg-2(qd101) |
| GR2098 | mgEx790[myo-3p::gfp::3′utr(32% GCinsert)]; smg-6(r896) |
| GR2099 | mgEx791[myo-3p::gfp::3′utr(60% GCinsert)]; |
| | myo-2::(nls)mcherry |
| GR2100 | mgEx791[myo-3p::gfp::3′utr(60% GCinsert)]; |
| | myo-2::(nls)mcherry; smg-1(r861) |
| GR2101 | mgEx791[myo-3p::gfp::3′utr(60% GCinsert)]; |
| | myo-2::(nls)mcherry; smg-2(qd101) |
| GR2102 | mgEx791[myo-3p::gfp::3′utr(60% GCinsert)]; |
| | myo-2::(nls)mcherry; smg-6(r896) |
| GR2103 | mgEx792[myo-3p::gfp::3′utr(70% GCinsert)]; |
| | myo-2::(nls)mcherry |
| GR2104 | mgEx792[myo-3p::gfp::3′utr(70% GCinsert)]; |
| | myo-2::(nls)mcherry; smg-1(r861) |
| GR2105 | mgEx792[myo-3p::gfp::3′utr(70% GCinsert)]; |
| | myo-2::(nls)mcherry; smg-2(qd101) |
| GR2106 | mgEx792[myo-3p::gfp::3′utr(70% GCinsert)]; |
| | myo-2::(nls)mcherry; smg-6(r896) |

Genetic and Mosaic Analysis of Mbl-1 Molecular Association with Expanded CUG Repeats

For genetic and mosaic analysis of mbl-1, C. elegans strains were generated expressing mbl-1 fused to the fluorophore mCherry for in vivo visualization. The strains generated expressing MBL-1::mCherry in C. elegans body wall muscles of an otherwise wild type animal exhibited a diffuse cellular distribution, with nuclear enrichment (shown in FIG. 8A-C). The MBL-1::mCherry strain was crossed with the 123CUG and 0CUG strains. The localization of the GFP mRNAs containing 123CUG repeats or the control with no repeats (0CUG) was analyzed by SM-FISH in the strain also expressing MBL-1::mCherry (results shown in FIGS. 1F and G, FIG. 8D). SMFISH followed by computational analysis of these images was performed to examine whether an increase in MBL-1 levels caused an increase in expanded RNA foci size or number. As a control, strains expressing isolated mCherry protein and 123CUG were also analyzed to test whether any increase in size or number of foci relative to the 123CUG strain was detected.

Mosaic analysis of GFP fluorescence intensity was also performed for muscle cells that expressed mbl-1::mCherry in a 123CUG background with no GFP fluorescence detected translated from the mRNAs bearing 123CUG repeats in their 3′UTR. Neighboring cells, that failed to express the mbl-1::mCherry transgene, were also analyzed for GFP signal and fluorescence was detected translated from the GFP mRNA bearing 123CUG repeats (results shown in FIG. 8E) and GFP fluorescence was similar to a strain that does not carry mbl-1::mCherry. As a control, strains expressing isolated mCherry protein and 123CUG were also analyzed to test whether the observed change in GFP fluorescence intensity was caused by mCherry protein expression in muscle cells.

### RNA Fluorescence In Situ Hybridization (RNA FISH)

Oligonucleotide probes were designed and SM-FISH was performed as described in Raj et al. (2008) Nat Methods. 5:877-9. SM-FISH was performed in 3d adult animals, and in human fibroblast cells 24 hour post siRNA transfection, using probes synthesized by BioSearch Technologies. Two probe sets were used for C. elegans samples, each with thirty-four probes complementary to gfp. One set of probes used was labeled with the dye CAL Fluor Red 590, and the other set with Quasar 670. A distinct probe set was used for the fibroblast cell samples, comprised of twenty-eight probes, labeled with the CAL Fluor Red 590 dye and targeting the CUG repeat region and the 3′ region of the dmpk mammalian gene (see Supplementary Notes). DAPI was used for nuclear staining and SM-FISH images were collected with an Olympus FV-1000 confocal microscope with an Olympus PlanApo 60 3 Oil 1.45 NA objective at 4 zoom, and a 559 nm (mCherry/CALFluor probe), 635 nm (Quasar probe) and 405 nm (DAPI) diode laser.

### SM-FISH Computational Image Analysis

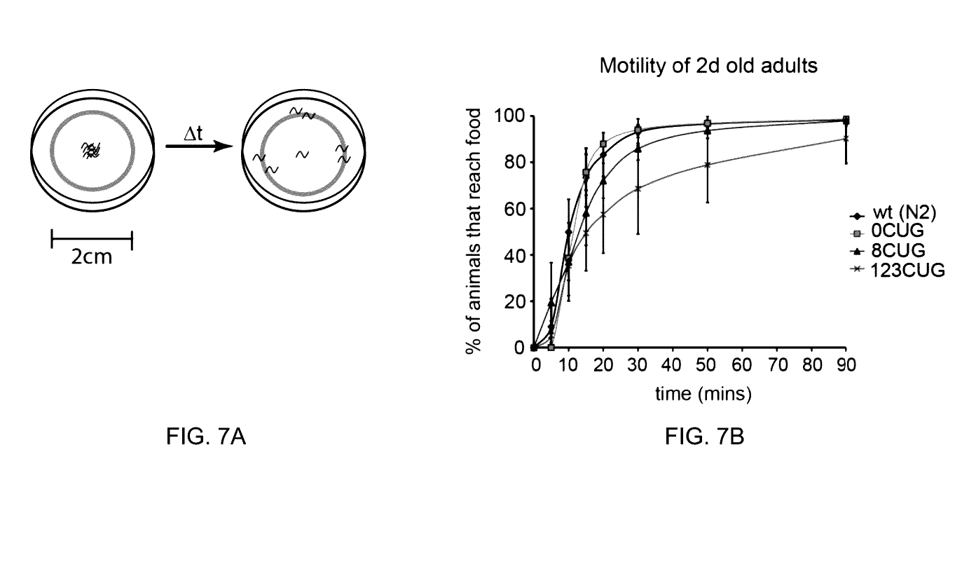

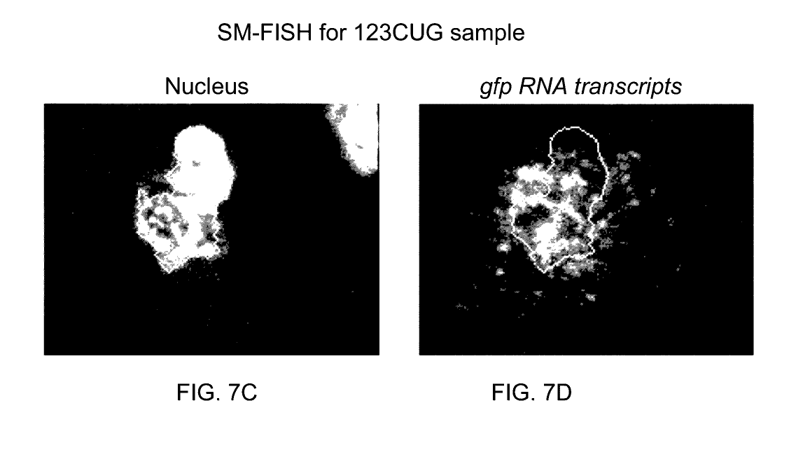

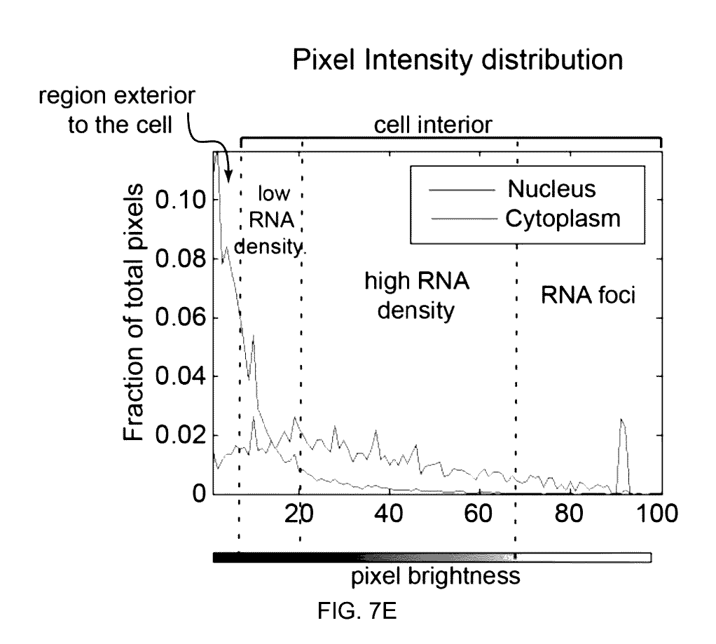

To analyze SM-FISH images, an algorithm was developed to quantify the RNA intensity pixel by pixel in the image. Based on its intensity, each pixel was categorized into one of three RNA populations present in the cell: ‘single’ RNAs (low RNA density), several RNA transcripts (high RNA density), and RNA foci structures (FIG. 7E). Pixel intensity corresponding to fluorescence intensity correlates with the number of RNA transcripts present. DAPI staining was used to identify the nucleus in each cell. Because the accumulation of foci in DM is characterized by its nuclear localization (asymmetric cellular foci distribution), the cytoplasmic region in each image was utilized to normalize for variations in staining. This approach would allow also the detection of changes in nuclear foci accumulation. This algorithm allowed us to calculate for each nucleus the percent of foci (pixels) and of “high density RNA” (pixels) from the total pixel population. The data was plotted where each ‘dot’ represents a nucleus, with the Y axis representing the percentage of foci pixels and the X axis indicating the percentage of pixels with ‘high density’ RNA.

C. elegans Fluorescence Imaging

For in vivo imaging, animals were mounted on a 2% agar pad on a glass slide and immobilized in 1 mg/ml levamisole (Sigma). Fluorescence imaging was done on a Zeiss AxioImager.Z1 Microscope.

### RNAi Screens

RNAi-mediated gene inactivation was by feeding in a 12-well plate RNAi bacterial culture 2× concentrated. Animals were synchronized by NaOCl bleaching and overnight hatching in M9. Twenty to thirty L1 larval stage animals (approximately 24 hours after synchronization) were aliquoted onto agar plates containing a 48 hour culture of RNAi bacteria expressing double-stranded RNA, and allowed to develop to adulthood. The drug 5-fluorodexoyuridine was added at the L4-larval stage to a final concentration of 0.1 mg/ml, to inhibit progeny production. Each 12-well plate contained the empty L4440 control vector as a negative control. Animals were analyzed either as 3d and as 4d old adults for the GFP fluorescence screen, or at 2d old adults for the locomotion-based toxicity screen. The RNAi clones identified as positives from the screen were verified by sequencing of the insert.

C. elegans Locomotion Assays

The locomotion assay on plates with a ring of OP50 food attractant was performed as previously described. The percentage of age-synchronized animals that reached the OP50 food in 90 minutes was determined. The second locomotion assay, with analysis of animal velocity, was performed at room temperature and off food. Each experiment performed contained a control corresponding to 123CUG and 0CUG animals fed on control vector (L4440). The locomotion behavior was recorded on a Zeiss Discovery Stereomicroscope using Axiovision software. The center of mass was recorded for each animal on each video frame using object-tracking software in Axiovision. Imaging began 30 minutes after animals were removed from food and recordings were 30 seconds long. For each assay, 20-45 2d old age-synchronized animals were recorded. The motility data was analyzed using the two-sample Kolmogorov-Smirnov test to compare the distributions of the values in the two data vectors x1 and x2. The null hypothesis is that x1 and x2 are from the same continuous distribution. This test was applied in two different ways 1) using the median velocities of all experiments obtained from all the 123CUG or 0CUG animals fed on control vector and 2) using the experimental internal control corresponding to the median velocity of the 123CUG or 0CUG on control vector. RNAi clones were only considered positive if strongly significant on both analyzes.

qRT-PCR

Total RNA was isolated from synchronized 2d old C. elegans adults using Trizol (Invitrogen) followed by chloroform extraction and isopropanol precipitation. Samples were DNase treated with Turbo DNA-free (Invitrogen) and cDNA was synthesized from 1 μg total RNA using Retroscript (Invitrogen). Quantitative RT-PCR assays of mRNA (SYBR Green, Bio-Rad) levels were done according to Bio-Rad recommendations. Three independent biological samples were used for all strains analyzed for gfp levels, and we used rpl-32 levels for normalization across samples. The 2-ΔΔct method was used for comparing relative levels of mRNAs.

### Protein Blot Assays

Proteins were extracted from synchronized animals and actin levels were used for normalization across samples. Three independent biological samples were used for all strains analyzed. Harvested C. elegans samples were boiled for 10 minutes in Laemmli buffer, spun and the supernatant collected. Proteins were resolved on 4-12% Bis-Tris SDS polyacrilamide gels, transferred to nitrocellulose membranes and probed with GFP and actin antibodies (Roche, Cat#11814460001; Abcam, ab3280). Protein levels were quantified on a Typhoon phosphoimager using the ImageQuant TL software (GE Healthcare Life Sciences). p values were calculated using Student's t test.

### Mammalian Cell Culture

Human lymphoblast cell lines were obtained from the Coriell Cell repository corresponding to cells from unaffected individuals (GM07492) and fibroblast from DM1-affected individuals (GM03989). Cells were maintained in high glucose EMEM (Lonza) supplemented with 15% fetal bovine serum, lx antibiotic-antimycotic (Gibco) and 1× non-essential amino acids solution (Sigma), at 37° C., 5% CO2.

siRNA Knockdown of UPF1 in Human Cells

Fibroblast cells were transfected with UPF1 ON-TARGETplus SMARTpool siRNA (Thermo Scientific, cat. No. J-011763-05), or nontargeting siRNA as control (Thermo Scientific, cat. No. D-001810-01) for 24 hours, using Lipofectamine RNAiMAX (Invitrogen) according to the manufacturer's protocol. The final siRNA concentration used was 100 nM. Cells were fixed after transfection for analysis by FISH as described in Raj et al. (2008) Nat Methods. 5:877-9. Knockdown efficiency was monitored by Western Blotting with a UPF1 and GAPDH specific antibodies.

### Foci Quantification in Human Fibroblasts

Nuclear foci in DM1-affected fibroblasts were quantified using the CellProfiler software, and specifically a script in CellProfiler, “Speckle Counting’ that allows the identification of individual cells, their nuclei, together with the number of foci present. The percentage of DM1 cells containing different numbers of nuclear foci was plotted and the p value calculated using two sample t-test function in the Matlab package.

### Example 1.1

### Expanded CUG Repeats Cause C. elegans Muscle Defects

A set of C. elegans reporter genes expressing GFP with 3′UTR containing various lengths of CTG repeats in body wall muscle cells was generated using the myo-3 muscle-specific promoter (FIG. 1A). Reporter constructs without any CUG repeats in the 384-nt 3′UTR from the let-858 gene (0CUG) displayed strong GFP fluorescence at all developmental stages, with a modest decline during adulthood. Analogous constructs with eight CUG repeats showed similar results with mild changes in GFP fluorescence. In contrast, the presence of 123 CUG repeats in the 3′UTR (123CUG, a pathogenic repeat length in mammalian myocytes) resulted in a sharp decline in GFP fluorescence as animals developed to adults. Western blotting analyses revealed a sharp decrease in GFP protein levels in 3 day (3d) old adult stage animals of the 123CUG strain (12% compared to protein levels at the L2 larval stage). The 3d adult stage animals of control 0CUG strain showed 50% of the GFP levels in L2 (FIG. 1B). The decline in adult stage GFP fluorescence in 123CUG transgenic animals was used for RNAi screens to identify genes that influence toxicity of expanded CUG repeats.

The function of C. elegans muscle expressing CUG repeats was investigated by assessing locomotion phenotypes of these animals. Motor defects were quantified by determining the percentage of animals that reached an attractant E. coli food ring (2 cm radius) on an agar plate in 90 minutes (FIG. 1C and FIG. 7A). The 123CUG strains exhibited severe motility deterioration at 6d adulthood, moving about five fold slower than wild type or control transgenic animals carrying 8CUGs or 0CUG constructs, which were similar to wild type. Synchronized populations of 123CUG animals at the 2d adult stage (FIG. 7B) and at the L4 stage also exhibited earlier locomotion defects, whereas strains bearing 8CUG or 0CUG repeats showed no motility defects. Thus, expanded CUG repeats cause progressive muscle dysfunction as C. elegans ages, as in other organisms including mammals.

Because nuclear inclusions of expanded CUG repeat RNAs are characteristic of myotonic dystrophy (DM), assessments were made as to whether 123CUG RNA transcripts formed nuclear foci in C. elegans muscle cells. Single molecule RNA fluorescence in situ hybridization (SM-FISH) was used which had higher sensitivity and specificity than traditional FISH16. The repeat-containing region of the expanded RNA transcript is known to interact inappropriately with RNA-binding proteins. Therefore RNA probes complementary to the GFP sequence were chosen because they are expected to be accessible in SM-FISH. SM-FISH detected the accumulation of expanded mRNA transcripts in foci as ‘large’, often amorphous, bright fluorescent structures, with 123CUG repeats mRNAs causing the accumulation of 2 to 5 nuclear foci per cell (FIG. 1D). Many individual fluorescence spots, likely corresponding to individual mRNAs, were also observed in the nucleus in the 123CUG strain (FIG. 1D). In contrast, animals expressing 0CUG or 8CUG repeat RNA transcripts lacked multiple bright nuclear foci, and exhibited a predominantly cytoplasmic distribution of RNA ‘single’ transcripts (FIG. 1D).

For a systematic analysis of all SM-FISH data to quantify foci formation and nuclear versus cytoplasmic RNA distribution for 123CUG repeats vs controls, an algorithm was developed that analyzed pixel intensity and cellular distribution in SM-FISH images (FIG. 7C-E). The SM-FISH images collected for the nuclear versus cytoplasmic distribution of CUG repeat RNA transcripts were examined as foci or as ‘concentrated single transcripts’ (high RNA density areas) (FIG. 7C-E). Consistent with the SM-FISH images (FIG. 1D), the analysis of multiple 123CUG images showed a higher nuclear fluorescence intensity, corresponding to nuclear foci and ‘single’ RNA transcripts (FIG. 1E), clearly distinct from the control 0CUG samples. The quantitative analysis also distinguished the 8CUG from the 0CUG samples, indicating that there are fewer RNA transcripts in the nucleus of 8CUG animals compared to 0CUG strains.

The mammalian splicing protein MBNL1 binds to RNA transcripts containing expanded CUG repeats, and in myotonic dystrophy, is sequestered by expanded CUG foci. SM-FISH and mosaic analysis in vivo were utilized to determine whether the C. elegans MBNL1 orthologue, MBL-119, bound the 123CUG foci detected in muscle cells. Expression of mbl-1 in a 123CUG background caused a marked increase in foci size relative to the 123CUG strain alone (FIGS. 1F and G, FIG. 8A-D). Mosaic analysis showed that MBL-1 caused the retention of expanded CUG repeat RNA transcripts in large nuclear foci disrupting transport to the cytoplasm and GFP translation (FIG. 8E). These effects were not observed with GFP mRNAs with 0CUG in a strain expressing MBL-1. Thus, as in other organisms, MBL-1 interacts in vivo with expanded CUG transcripts in C. elegans, and MBL-1 association with expanded CUG repeat transcripts decreases mRNA export to the cytoplasm and translation. Down-regulation of mbl-1 by RNAi did not disrupt or enhance 123CUG transcript foci accumulation (FIG. 8F). MBL-1 down-regulation, can affect the levels of expanded CUG transcript available for translation. These data suggested that additional regulatory factors contribute to expanded CUG foci accumulation and toxicity. Without wishing to be bound by theory, it is believed that the RNA aggregated transcripts identified by SM-FISH correspond to the key foci characteristic of DM.

### Example 1.2

### Screen for Modifiers of Expanded CUG-Mediated Toxicity

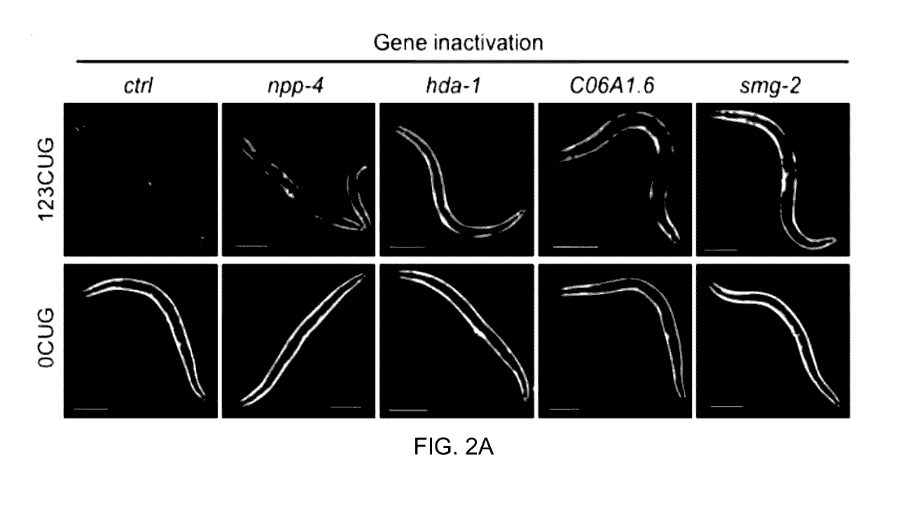

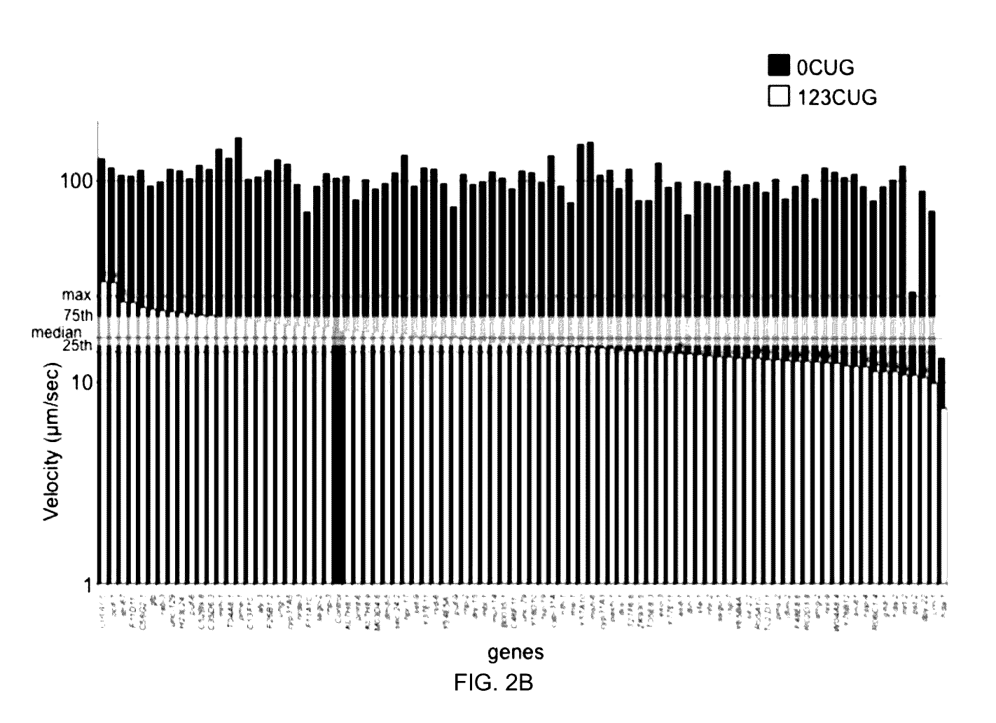

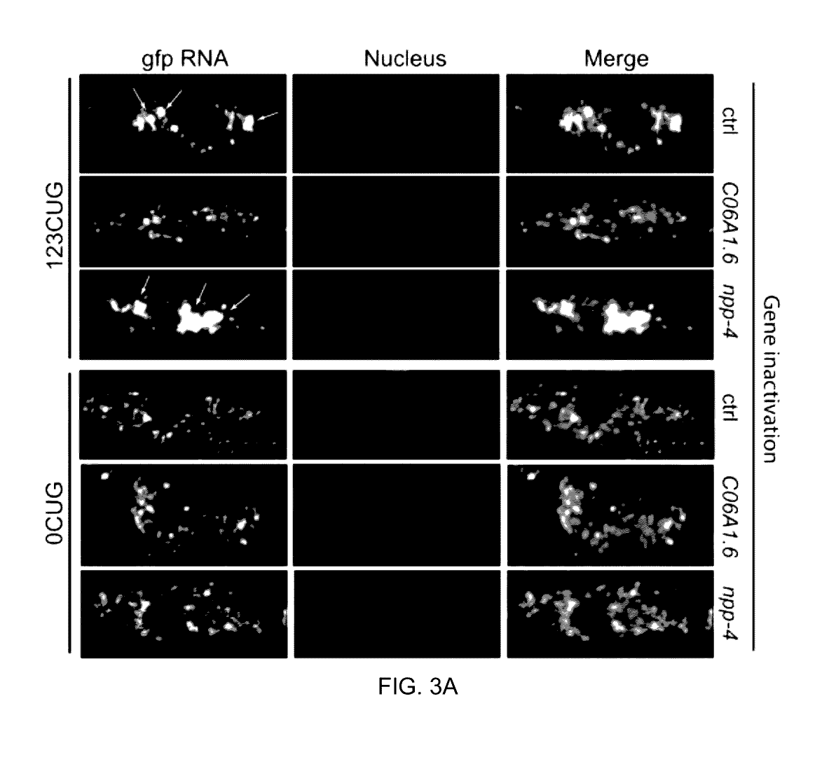

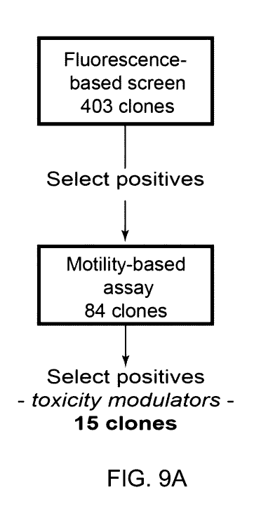

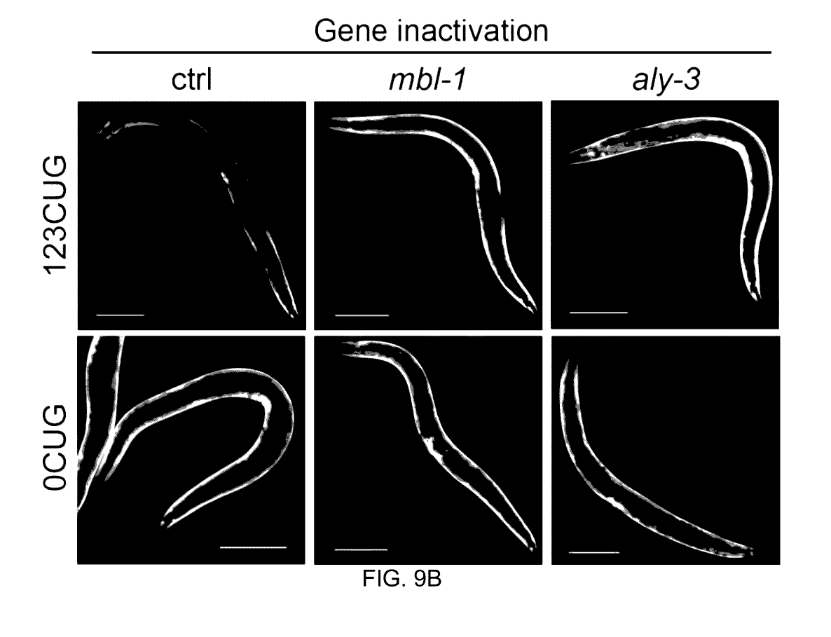

To identify genes that mediate expanded CUG repeat RNA pathogenesis, RNAi was used to reveal gene inactivations that can modify expanded CUG repeat RNA toxicity. A two-step screen was performed, with an initial fluorescent-based RNAi screen, followed by a secondary motility-based screen on hits from the primary screen (FIG. 9A). For the fluorescent-based screen, gene inactivations were assayed that disrupt the late stage down-regulation of GFP fluorescence specific to the 123CUG strain. An RNAi library of 403 clones targeting genes that encode RNA-binding proteins and factors implicated in small RNA pathways was screened. This type of sub-library was expected to have a high representation of genes involved in expanded CUG repeat toxicity. Of the 403 genes tested, after re-screening in triplicate, 84 gene inactivations were selected that induced an increase in late developmental stage GFP fluorescence specifically in the 123CUG strain without affecting the control 0CUG strain (FIG. 2A, FIG. 9B, Table 2).

Each of the 84 gene inactivations identified was tested for their ability to modulate the motility defect observed in 123CUG animals. The 123CUG animals on the control RNAi showed a severe loss in motility, with a median velocity of ≈17 μm/sec, compared to the 0CUG strain on the same control RNAi at ≈100 μm/sec (FIG. 2B) similar to wild type animals. Fourteen gene inactivations were identified that significantly (p<0.01 using the two-sample Kolmogorov-Smirnov test) increased or decreased the velocity of 123CUG animals without affecting the control (0CUG) animals (FIG. 2B, Table 3).

The list of genetic modifiers of expanded CUG toxicity identified can be categorized into the following three major classes: genes involved in transcription, signaling, and RNA processing and degradation (Table 3).

Some of the genes identified had been previously implicated in polyglutamine (polyQ) repeat disorders: the hda-2, mrt-2 and smg-2 genes, corresponding to a histone deacetylase, a RAD1 911 complex DNA damage checkpoint protein, and a RNA helicase part of the nonsense-mediated decay pathway, respectively. smg-2 was included in the final list as an additional gene inactivation that affected both the 123CUG repeat transgene and the 0CUG control transgene; smg-2 gene inactivation caused a mild decrease in motility of the 0CUG strain, but caused a much stronger loss of motility for 123CUG repeat strain and was the strongest hit from the fluorescent screen for suppression of the 123CUG-specific decline in GFP fluorescence. The identification in this screen of common regulators of expanded repeat diseases supports the view that repeat-associated disorders, where repeats occur in either coding or non-coding regions, share several protein cofactors.

TABLE 2

| | | | | Mammalian |
|---|---|---|---|---|
| Sequence | Gene | Gene description (function) | Category | orthologue |
| C53A5.3 | hda-1 | histone deacetylase 1 | Transcription | HDAC1 |
| F46G10.7 | sir-2.2 | Sirtuin 4, histone deacetylase | Transcription | |
| C08B11.2 | hda-2 | Histone deacetylase complex, | Transcription | HDAC1 |
| | | catalytic component RPD3 | | |
| Y65B4A.1 | | Transcription elongation factor | Transcription | HTATSF1 |
| | | TAT-SF1 | | |
| C52B9.8 | | Chromatin remodeling complex | Transcription | SMARCA2 |
| | | SWI/SNF, component SWI2 | | |
| R06C7.7 | lin-61 | Polycomb group protein | Transcription | SFMBT1 |
| | | SCM/L(3)MBT | | |
| C32F10.6 | nhr-2 | nuclear hormone receptor | Transcription | NR1D1 |
| F15E6.1 | set-9 | PHD Zn-finger protein; Histone- | Transcription | SETD5 |
| | | lysine N-methyltransferase (relieves | | |
| | | transcriptional repression) | | |
| C53D6.2 | unc-129 | member of the TGF-beta family of | Transcription | BMP3 |
| | | secreted growth factor signaling | | |
| | | molecules | | |
| F47A4.2 | dpy-22 | Thyroid hormone receptor- | Transcription | MED12L |
| | | associated protein complex, subunit | | |
| | | TRAP230 | | |
| F10C1.5 | dmd-5 | Transcription factor Doublesex | Transcription | DMRTB1 |
| C23H5.1 | prmt-6 | Protein arginine methyltransferases | Transcription | COQ3 |
| Y56A3A.29 | ung-1 | uracil-DNA glycosylase, required for | Replication, recombination | UNG |
| | | genomic stability | and repair | |
| F32A11.2 | hpr-17 | Cell cycle checkpoint, | Replication, recombination | RAD17 |
| | | RAD17-RFC complex | and repair | |
| R09B3.1 | exo-3 | Apurinic/apyrimidinic endonuclease | Replication, recombination | APEX1 |
| | | | and repair | |
| Y47G6A.8 | crn-1 | no gene name - 5′-3′ exonuclease | Replication, recombination | FEN1 |
| | | | and repair | |
| Y47G6A.11 | msh-6 | Mismatch repair ATPase MSH6 | Replication, recombination | MSH6 |
| | | | and repair | |
| H12C20.2 | pms-2 | DNA mismatch repair protein | Replication, recombination | PMS2 |
| | | | and repair | |
| R10E4.5 | nth-1 | endonuclease III-like | Replication, recombination | NTHL1 |
| | | | and repair | |
| Y57A10A.j | | 3′-5′ exonuclease | Replication, recombination | |
| | | | and repair | |
| Y41C4A.14 | mrt-2 | Checkpoint 9-1-1 complex, RAD1 | Replication, recombination | RAD1 |
| | | component | and repair | |
| R02D3.8 | | exonuclease | Replication, recombination | ERI3 |
| | | | and repair | |
| T28A8.7 | mlh-1 | DNA mismatch repair protein | Replication, recombination | MLH1 |
| | | | and repair | |
| Y71F9AL.18 | pme-1 | NAD+ ADP-ribosyltransferase Parp | Replication, recombination | PARP1 |
| | | | and repair | |
| C06A1.6 | | endonuclease | Transcription | KRTAP5-7 |
| | | | Replication, recombination | |
| | | | and repair | |
| R74.5 | asd-1 | ataxin 2-binding protein; alternative | RNA processing and | RBFOX3 |
| | | splicing component | modification | |
| K09B11.2 | nol-9 | Uncharacterized conserved protein | RNA processing and | NOL9 |
| | | similar to ATP/GTP-binding protein | modification | |
| F29C4.7 | grid-1 | Large RNA-binding protein | RNA processing and | RBM15 |
| | | | modification | |
| Y116A8C.32 | sfa-1 | Splicing factor 1/branch point | RNA processing and | SF1 |
| | | binding protein | modification | |
| K08D10.4 | rnp-2 | Spliceosomal protein snRNP- | RNA processing and | SNRPB2 |
| | | U1A/U2B | modification | |
| R10E9.1 | msi-1 | mRNA cleavage and polyadenylation | RNA processing and | MSI2 |
| | | factor I complex, subunit HRP1 | modification | |
| R06C1.4 | | mRNA cleavage and polyadenylation | RNA processing and | CSTF2T |
| | | factor I complex | modification | |
| Y113G7A.9 | dcs-1 | Scavenger mRNA decapping | RNA processing and | DCPS |
| | | enzyme | modification | |
| T05E8.3 | | DEAH-box RNA helicase | RNA processing and | DHX33 |
| | | | modification | |
| K07H8.9 | | RNA-binding protein Sam68 | RNA processing and | QKI |
| | | | modification | |
| C46F11.4 | | ATP-dependent RNA helicase | RNA processing and | DDX42 |
| | | | modification | |
| K08D10.3 | rnp-3 | Spliceosomal protein snRNP- | RNA processing and | SNRPA |
| | | U1A/U2B | modification | |
| D2089.2 | rsp-7 | Splicing factor, arginine/serine-rich | RNA processing and | MARCH5 |
| | | | modification | |
| D1046.1 | cfim-2 | mRNA cleavage factor I | RNA processing and | CPSF6 |
| | | subunit/CPSF subunit | modification | |
| M18.7 | aly-3 | | RNA processing and | THOC4 |
| | | | modification | |
| F11A10.2 | repo-1 | Splicing factor 3a, subunit 2 | RNA processing and | SF3A2 |
| | | | modification | |
| F11A10.7 | | nucleolar protein | RNA processing and | NCL |
| | | | modification | |
| B0035.12 | | RNA-binding protein SART3 | RNA processing and | SART3 |
| | | | modification | |
| F26B1.2 | | Heterogeneous nuclear | RNA processing and | HNRNPK |
| | | ribonucleoprotein k | modification | |
| K07H8.10 | | nucleolin | RNA processing and | NCL |
| | | | modification | |
| Y54E5A.4 | npp-4 | Nuclear pore complex component, | RNA transport | NUPL1 |
| | | nucleoporin | | |
| F16D3.2 | rsd-6 | spreading defective factor | Small RNA pathways | SPEN |
| C14C11.6 | mut-14 | ATP-dependent RNA helicase | Small RNA pathways | DDX3X |
| R04A9.2 | nrde-3 | Argonaut protein | Small RNA pathways | AGO1 |
| M03D4.6 | | Translation initiation factor 2C | Small RNA pathways | AGO4 |
| F56A6.1 | sago-2 | Argonaute homolog | Small RNA pathways | AGO1 |
| K12B6.1 | sago-1 | Argonaute homolog | Small RNA pathways | AGO4 |
| C35D6.3 | | Unnamed protein; uncharacterized | Small RNA pathways | |
| T22A3.5 | pash-1 | | Small RNA pathways | DGCR8 |
| F07A11.6 | din-1 | | Small RNA pathways | ZC3H13 |
| F18A11.1 | puf-6 | Translational repressor | Translation, ribosomal | PUM |
| | | Pumilio/PUF3 and related RNA- | structure and biogenesis | |
| | | binding proteins | | |
| Y54E5A.6 | | tRNA-dihydrouridine synthase | Translation, ribosomal | DUS2L |
| | | | structure and biogenesis | |
| W06B11.2 | puf-9 | Translational repressor | Translation, ribosomal | PUM2 |
| | | Pumilio/PUF3 | structure and biogenesis | |
| F48E8.6 | | Exosomal 3′-5′ exoribonuclease | Translation, ribosomal | DIS3L2 |
| | | complex, subunit Rrp44/Dis3 | structure and biogenesis | |
| K08D10.2 | dnj-15 | heat shock DNaJ protein | Protein Folding | HSCB |
| ZC518.2 | sec-24.2 | Vesicle coat complex COPII, subunit | Protein Transport | SEC24B |
| | | SEC24/subunit SFB2 | | |
| F54E7.1 | pst-2 | C. elegans ortholog of the PAPST2 | Protein Transport | SLC35B3 |
| | | PAPS (3′-phospho-adenosine-5′- | | |
| | | phosphosulfate) transporter | | |
| E03A3.6 | unc-79 | alpha-1 subunits of voltage- | Neuronal signaling | UNC79 |
| | | insensitive cation leak channels | | |
| C18A3.6 | rab-3 | member of the Ras GTPase | Neuronal signaling | RAB3C |
| | | superfamily; GTPase Rab3, small G | | |
| | | protein superfamily | | |
| C16C2.3 | ocrl-1 | inositol-1,4,5-triphosphate 5- | Signaling | OCRL/ |
| | | phosphatase homolog | | INPP5B |
| D2092.7 | tsp-19 | | Signaling | SGPP1 |
| T27F6.6 | | | Signaling | SMPD2 |
| C56G2.1 | | Kinase anchor protein AKAP149 | Signaling | AKAP1 |
| K10C9.6 | str-67 | 7-transmembrane olfactory receptor | Signaling | OR4F5 |
| H23L24.4 | | Unnamed protein | Signaling | BRS3 |
| H02112.8 | cyp-31A2 | Cytochrome P450 | Metabolism | |
| | | CYP4/CYP19/CYP26 subfamilies | | |
| Y77E11A.7 | | exokinase | Metabolism | |
| Y17G9B.3 | cyp-31A3 | Cytochrome P450 family | Metabolism | CYP4V2 |
| Y62E10A.15 | cyp-31A5 | Cytochrome P450 family | Metabolism | CYP4V2 |
| T04A8.13 | | neurofilament triplet M domain | uncharacterized | MAP1B |
| R05A10.1 | | Unnamed protein | uncharacterized | ADCY4 |
| F11D11.3 | | Unnamed protein | uncharacterized | TTN |
| C13F10.5 | | Unnamed protein | uncharacterized | SAYSD1 |
| Y18D10A.8 | | Unnamed protein | uncharacterized | PRR12 |
| ZK930.5 | | Unnamed protein | uncharacterized | RP1 |
| Y37E11A_93.f | | | uncharacterized | |
| W04A8.4 | | 3′-5′ exonuclease | uncharacterized | — |
| T02D1.1 | | transposon | — | — |
| Y76B12C.5 | | transposon | — | — |

TABLE 3

| | | | | | | | Relative | |
|---|---|---|---|---|---|---|---|---|
| | | | | | | | Velocity as a | |
| | | | | | | | percentage | RNA foci |
| | | | | | | | of 123CUG | relative to |
| | Gene | | | | Human | | on cqf | 123CUG |
| | inactivation | Gene | Molecular Function | Class | ortholog | Motility | vector | alone |
| Toxicity | K10C9.6 | str-67 | G-protein coupled | Signaling | OR4F5 | improved | 148 | decrease |
| Enhancer | | | receptor | | | | | |
| | C16C2.3 | ocrl-1 | inositol-1,4,5-triphosphate | Signaling | OCRL | improved | 184 | mild |
| | | | 5-phosphatase | | | | | decrease |
| | C06A1.6 | | uncharacterized | Cytoskeleton | KRTAP5-7 | improved | 187 | decrease |
| | | | | homology | | | | |
| | R05A10.1 | | uncharacterized | Signaling | ADCY4 | worsened | 78 | increase |
| | K09B11.2 | nol-9 | polynucleotide 5′hydroxyl- | RNA | NOL9 | worsened | 74 | increase |
| | | | kinase (nucleolar protein) | Processing | | | | |
| | Y48G8AL.6 | smg-2 | helicase | RNA Processing | UPF1 | worsened | 75 | increase |
| | | | | and Degradation | | | | |
| | Y54E5A.4 | npp-4 | nuclear pore complex | RNA Transport | NUPL1 | worsened | 70 | increase |
| | | | protein | | | | | |
| | R74.5 | asd-1 | alternative splicing | RNA Processing | FOX2 | worsened | 82 | mild |
| | | | family member | | | | | increase |
| | F47A4.2 | dpy-22 | mediator complex subunit | Transcription | MED12L | worsened | 62 | mild |
| | | | transcriptional mediator | | | | | increase |
| | | | of RNA | | | | | |
| Toxicity | C08B11.2 | hda-2 | histoue deacetylase | Transcription | HDAC1 | worsened | 66 | decrease |
| Suppressor | Y41C4A.14 | mrt-2 | conserved DNA-damage | DNA Repair and | RAD1 | worsened | 65 | decrease |
| | | | checkpoint protein | Recombination | | | | |
| | F29C4.7 | grld-1 | RNA-binding protein | RNA Processing | RBM15B | worsened | 88 | mild |
| | | | (splicing) | | | | | decrease |
| | R06C1.4 | | uncharacterized | RNA Processing | CSTF2T | worsened | 88 | mild |
| | | | | and Degradation; | | | | decrease |
| | | | | Translation | | | | |
| | D1046.1 | cfim-2 | cleavage and | RNA Processing | CPFS7 | worsened | 76 | no change |
| | | | polyadenylation factor | and Degradation | | | | |
| | F48E8.6 | | ribonuclease | RNA processing | DIS3L2 | worsened | 75 | no change |
| | | | | and Degradation | | | | |

### Example 1.3

### CUG Toxicity Modulators Affect Nuclear Foci Accumulation

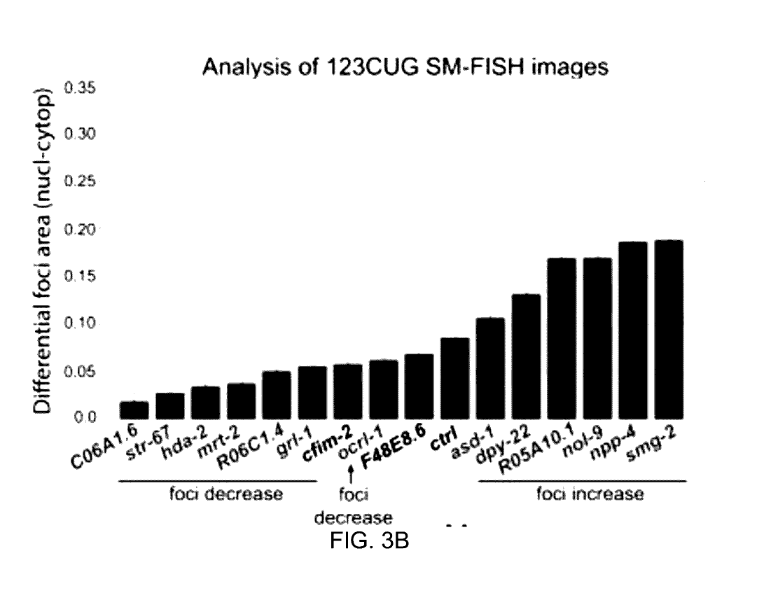

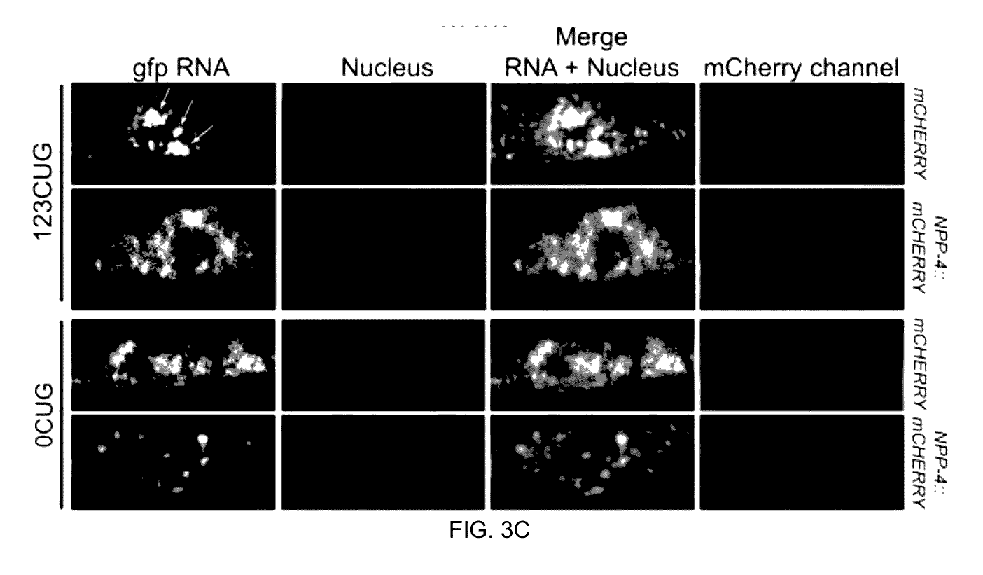

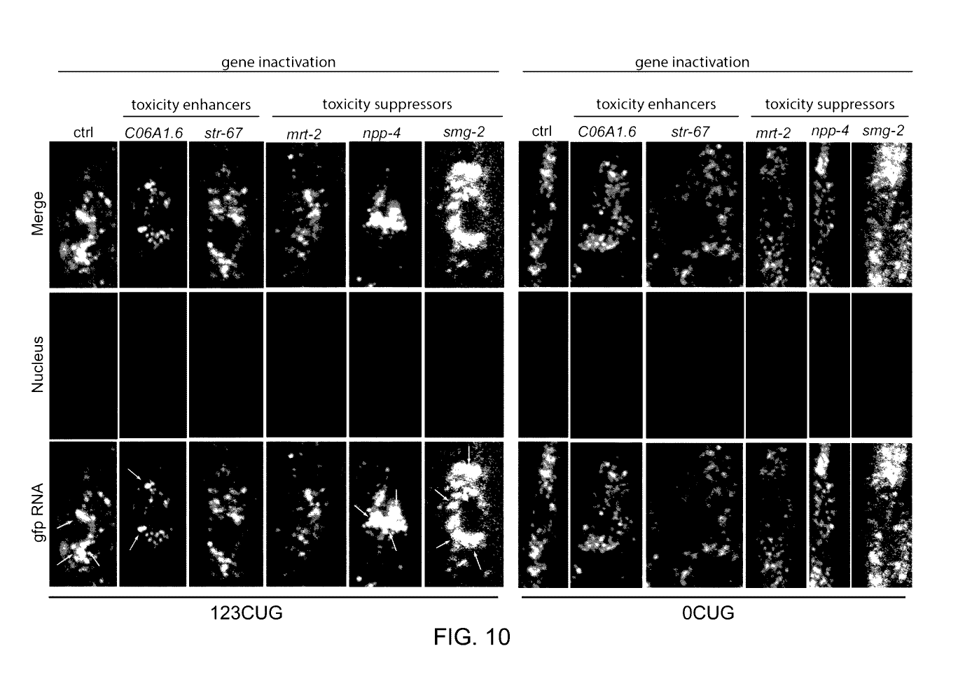

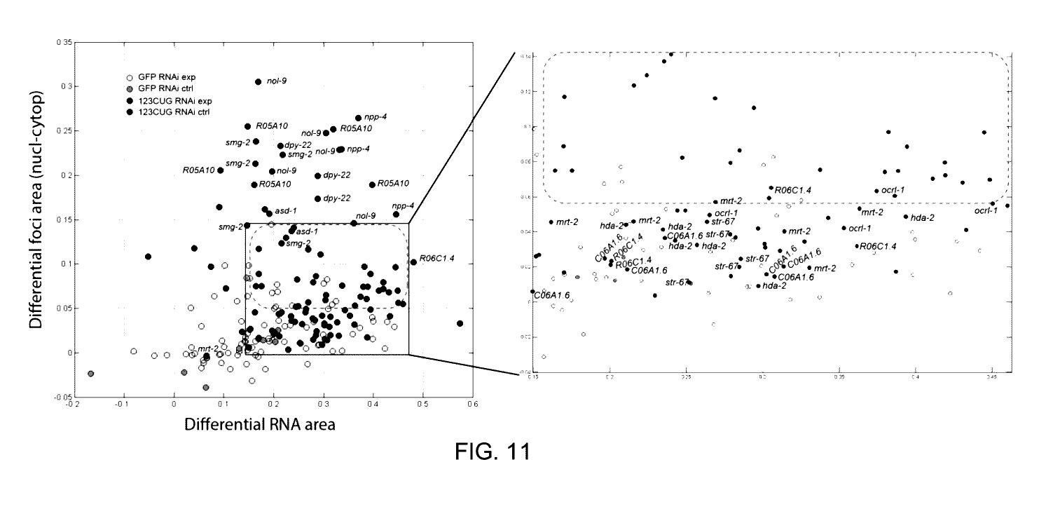

Experiments were carried out to determine whether any of the 15 gene inactivations that modulated expanded CUG repeat toxicity changed RNA foci accumulation of 123CUG transcripts. One prediction was that gene inactivations that improve the motility of animals expressing 123CUG RNAs would also cause a decrease in foci size or number and similarly, gene inactivations that caused further motility impairment would lead to an increase in foci size or number (Table 3). Of the 15 genes identified, inactivation of ocrl-1/inositol-1,4,5-triphosphate 5-phosphatase, str-67/GPCR chemoreceptor and C06A1.6, led to an improvement of motility in strains expressing 123CUG repeats in muscle (Table 3). Examination of GFP mRNA localization by SM-FISH in 123CUG muscles revealed a significant reduction in the number of nuclear foci when these three genes are inactivated (FIGS. 3A and B, FIGS. 10 and 11). The suppression of 123CUG foci was particularly striking for C06A1.6 gene inactivation, where 123CUG foci were now few and small, with SM-FISH signals close to 0CUG control levels (FIG. 3A, FIG. 10). However, distribution of expanded RNA ‘single’ transcripts was still observed preferentially in the nucleus versus the cytoplasm for all 3 gene inactivations, suggesting a role for ocrl-1, str-67 and C06A1.6 in foci formation rather than in cellular distribution of RNA. No significant changes in RNA localization, and no foci accumulation, were found in the control 0CUG strain, when these 3 genes were inactivated (FIG. 3A, FIGS. 10 and 11). Together, these data support a model in which ocrl-1, str-67 and C06A1.6 gene activities normally enhance the toxicity of expanded CUG repeats by contributing to 123CUG foci formation, and inactivation of these genes results in decreased toxicity.

For the 12 gene inactivations that further reduced motility in 123CUG animals, six gene inactivations caused an increase in foci size present in the nucleus of 123CUG body wall muscle cells. These genes are npp-4/nuclear pore complex protein, asd-1/alternative splicing regulator, smg-2/nonsense-mediated decay (NMD) factor, nol-9/polynucleotide 5′-hydroxyl-kinase, dpy-22/transcriptional mediator protein and R05A10.1 (FIG. 3, Table 3, FIGS. 10 and 11). For some genes, such as npp-4, a change in RNA localization was observed, with transcript enrichment in the nucleus relative to the cytoplasm (FIGS. 10 and 11). For all these genes, except smg-2, no significant changes in transcript distribution were observed for the control 0CUG mRNA. smg-2 gene inactivation in the control 0CUG led to a slight increase in transcript signal, in both the nucleus and cytoplasm, without affecting nuclear to cytoplasm RNA distribution or leading to foci formation. Inactivation of the other 6 genes either caused a reduction in foci sizes or did not cause a significant change in aggregate size or number (Table 3). The reduction of foci number associated with an increase in toxicity suggested that, in certain conditions, the accumulation of non-aggregated CUG-expanded RNAs can be a major contributor of cellular dysfunction. These ‘free’ toxic RNAs would have the potential to affect the activity of a wider range of RNA-binding proteins than when in an ‘aggregated’ state.

To further establish that the genes identified were involved in the regulation of expanded CUG-mediated toxicity, npp-4/nuclear pore complex component and asd-1/alternative splicing regulator as mCherry fusion proteins were overepxressed in body wall muscle cells in C. elegans. Down-regulation of npp-4 and asd-1 by RNAi caused an increase in nuclear expanded CUG RNA foci sizes (Table 3). C. elegans expressing these proteins fused to the fluorophore mCherry in either 123CUG or control 0CUG backgrounds, were analyzed by SM-FISH for a change in accumulation of 123CUG RNA in nuclear foci. Overexpressing either of these genes led to a decrease in foci number in a 123CUG background relative to the 123CUG parental strain (FIG. 12A). In contrast, overexpression of these proteins in the 0CUG strain had no effect on GFP mRNA transcript distribution. Expression of mCherry alone (FIG. 1F), or a different protein, such as RNP-2, had no effect on 123CUG foci size or number (FIG. 12A). Thus some of the genes identified are dosage sensitive components of the CUG repeat toxicity pathway.

Nonsense-Mediated Decay Targets 3′UTRs with CUG Repeats

Smg-2 RNAi in 123CUG animals caused an increase in nuclear RNA foci sizes, an increase in muscle cell toxicity with loss of motility and increase in GFP fluorescence signal relative to the control. smg-2 gene inactivation on control 0CUG strains had no effect on nuclear foci, and the mild increase in toxicity detected was not comparable to that observed in the 123CUG strain. In addition, smg-2 acts as a common regulator of expanded repeat-containing disorders by also suppressing protein aggregation caused by expanded CAG repeats in the coding regions of the Huntingtin gene, associated to Huntington's disease.

Smg-2 encodes an RNA helicase and is a conserved component of the nonsense-mediated mRNA decay (NMD) pathway. The NMD pathway is an evolutionary conserved surveillance mechanism that detects mRNAs containing premature stop codons, preventing toxic expression of truncated proteins. The identification of smg-2 as a modulator of expanded CUG toxicity suggested that the NMD pathway may recognize and target for degradation RNA transcripts with expanded CUG repeats, even in the 3′ UTRs of non-truncated open reading frames. The effects of mutations in NMD components on GFP transcripts bearing 123CUG repeats or control 0CUG in muscle cells were assessed using smg-1(r861), smg-2(qd101) and smg-6(r896) mutants. 123CUG animals in the background of any of the smg mutants showed a strong increase in GFP fluorescence signal relative to the parental strain (FIG. 4A). No such change in fluorescence was observed for the control 0CUG animals (FIG. 4A). Quantitative RT-PCR showed that, mRNA levels of gfp bearing 123CUG repeats were increased by several fold: ≈5.3 fold in smg-1(r861), ≈7.8 fold in smg-2(qd101) and ≈10.1 fold in smg-6(r896) backgrounds, compared to wild type (FIG. 4B). However, no significant change was observed in the levels of gfp mRNA without any CUG repeats in the 3′UTR in the different smg mutant backgrounds compared to the wild type (FIG. 4B). Thus the NMD pathway targets the mRNA transcripts containing the expanded CUG repeats for degradation.

SM-FISH and computational image analysis were utilized to analyze the gfp RNA transcript accumulation in 123CUG and the control 0CUG strains in the different smg mutant backgrounds. Disruption of NMD pathway in 123CUG animals caused an increase in foci size and number in the nucleus (FIG. 4C, FIG. 12B) and in most cells the accumulation of foci-like structures in the cytoplasm as well (FIGS. 4C and D, FIG. 12B). Conversely, in the smg mutant animals expressing the control 0CUG a uniform distribution of RNA transcripts was observed with a large number present preferentially in the cytoplasm (FIG. 4C, and FIG. 12B). Thus the NMD pathway recognizes RNA transcripts containing expanded CUG repeats and disruptions in NMD cause the accumulation of expanded CUG toxic RNA species in the nucleus, leading to cellular dysfunction (FIG. 14A).

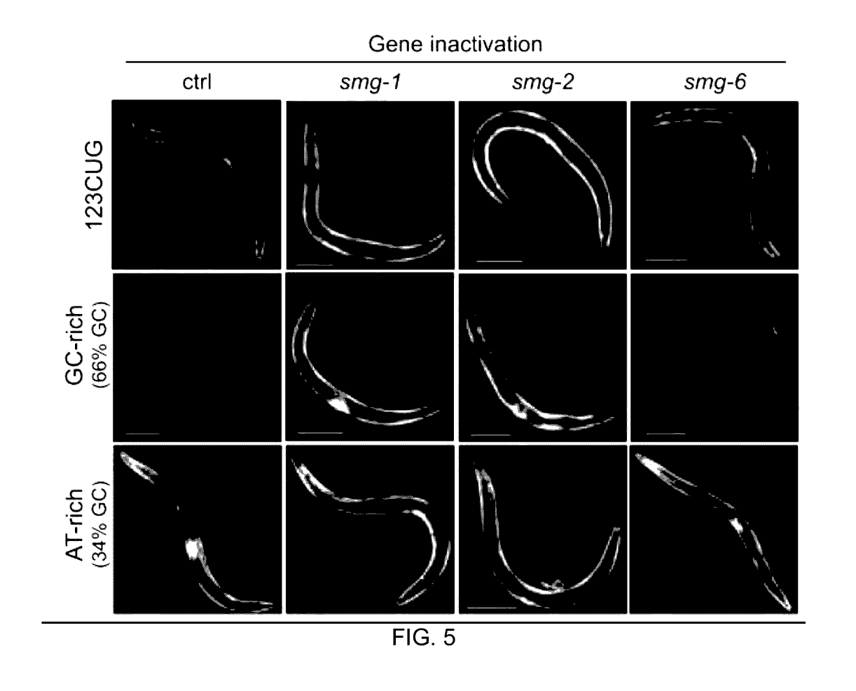

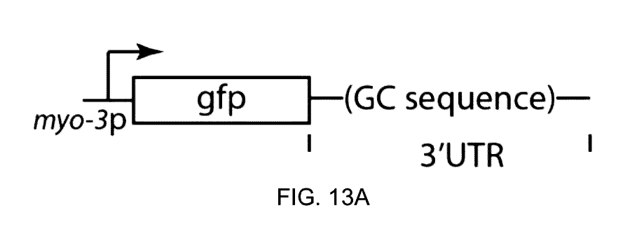

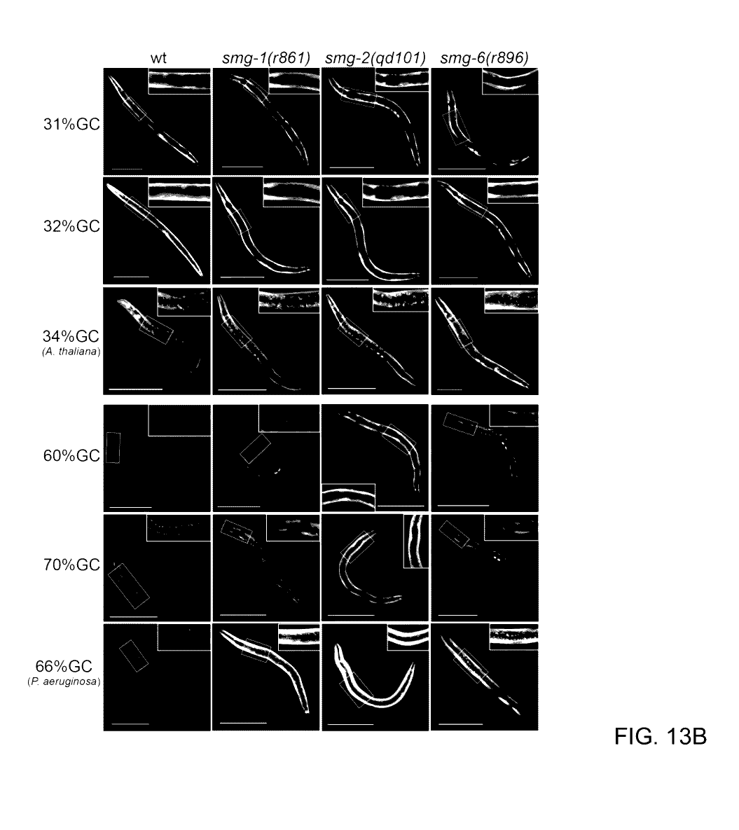

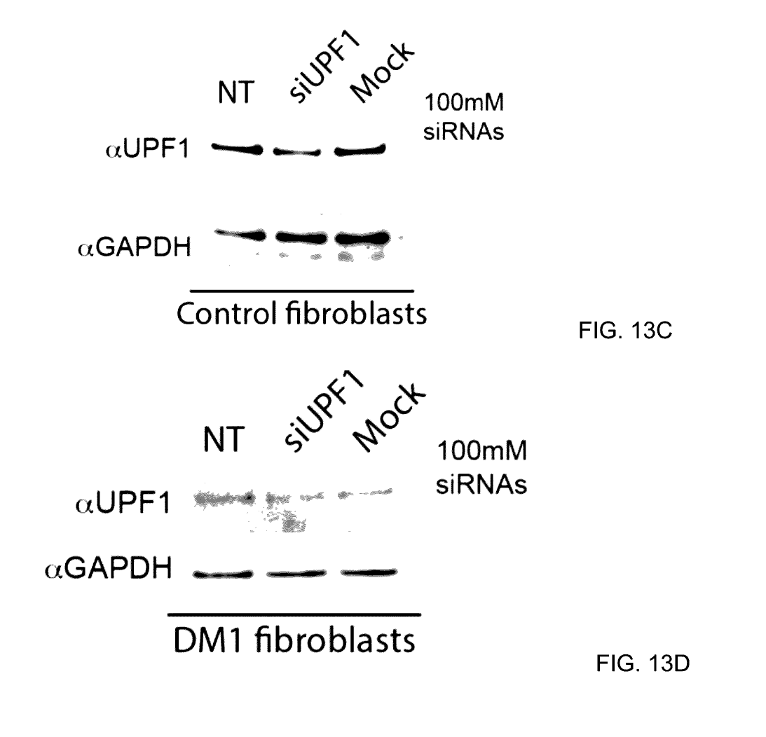

To examine whether the skewed sequence composition of expanded CUG repeat sequences targets them for the NMD pathway, the influence of GC composition on NMD was examined. 3 ‘UTRs are typically A/U rich (≈65-70% AT-rich), exhibiting a nucleotide composition distinct from coding (≈50-55% AT-rich) or intergenic regions. The let-858 3’ UTR to which the 123 CUG repeat was added is 384 nucleotides and 30% GC. The added CUG repeat elements are rich in G and C nucleotides (≈66%) that may contribute to the recognition by the NMD pathway. Expression plasmids were generated in which the 3′UTR (CTG)n sequence was substituted by a non-repeat sequence with either a 66% or 34% GC nucleotide content (FIGS. 13A and B). The DNA sequences used were cloned from non-C. elegans organisms or from entirely synthetic nucleotide sequences bearing similar GC percentages to avoid a possible recognition of endogenous signal sequences. GFP reporter genes bearing GC-rich 3′ UTR elements from non-C. elegans organisms exhibited weaker GFP fluorescence, or no fluorescence at all in the case of synthetic sequences, compared to those bearing the corresponding AT-rich elements (FIG. 5, FIG. 13B). Strains expressing GC-rich elements from a non-C. elegans genome placed in the 3′UTR of the GFP reporter gene showed a significant increase in fluorescence when either smg-1 or smg-2 were inactivated by RNAi, whereas no change in GFP intensity was detected for AT-rich (FIG. 5, FIGS. 13A and B). Fusion genes engineered with synthetic, random high GC percentage sequences showed a stronger increase in fluorescence in the smg-2 background relative to two regulators of smg-2 phosphorylation smg-1 or smg-6 (FIGS. 13A and B). These data demonstrate that the results observed for the GC-rich versus AT-rich sequences were not due to a sequence-specific endogenous 3′ UTR identity signal present in the sequence used. These results further suggest that the increase in distance between the stop codon and the polyA signal due to the addition of the CUG repeat sequence does not contribute to NMD recognition, since no repression was observed for AT-rich transcripts. These data support a model in which mRNAs, containing CUG repeats in their 3′UTR, are NMD substrates. Furthermore, the data reveals that the NMD recognition of CUG-containing mRNA is dependent on nucleotide composition, either due to the presence of a GC-rich sequence in a region usually A/U-rich, or due to the formation of specific secondary structures associated to the presence of these nucleotides. While both the GC-rich 3′ UTR element and the 123CUG repeat element reporter genes are responsive to disruption of the NMD pathway, none of the 15 gene inactivations that strongly disable 123 CUG repeat repression in muscle disrupt the repression conferred by GC-rich element. Thus, the detection and localization to foci of 123 CUG repeats by these genes is distinct from the detection and degradation of GC rich elements by the NMD system.

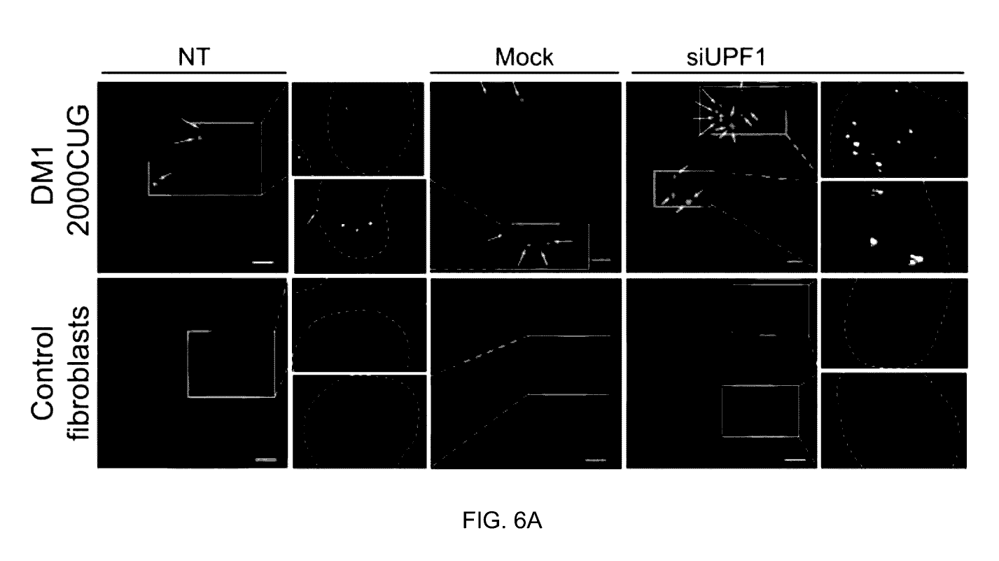

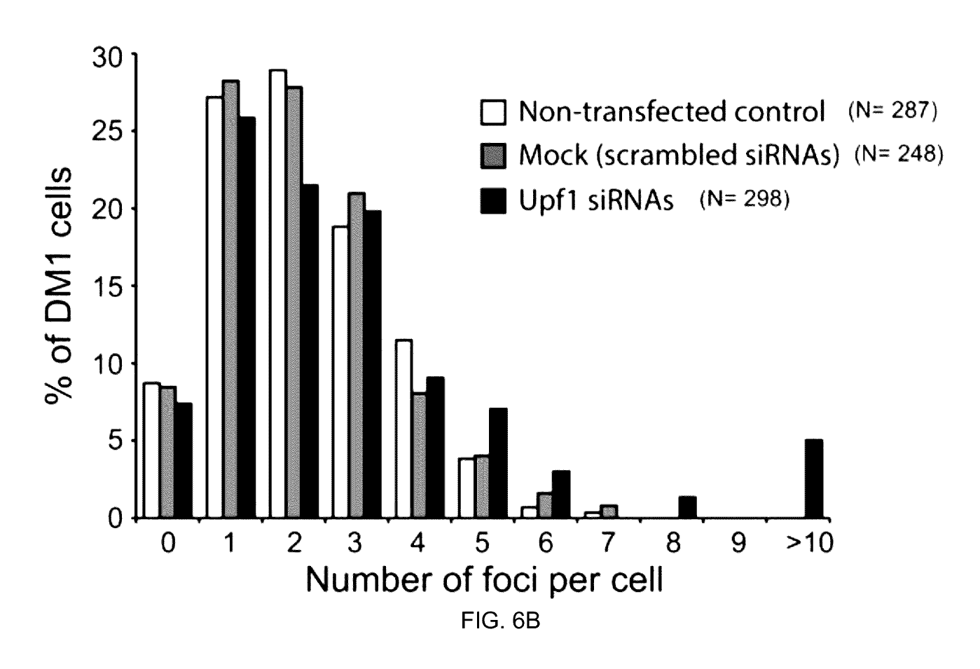

To establish whether NMD recognition of expanded CUG repeats is a conserved cellular mechanism, the nuclear RNA foci phenotype of NMD gene inactivations was examined in human DM1 patient fibroblast cells expressing 2000 CUG repeats in the DMPK1 mRNA, as well as in control fibroblasts expressing a DMPK1 mRNA with 7 to 35 such CUG repeats. Changes in foci number were tested when the human orthologue of smg-2, UPF1 was inactivated by RNAi. SM-FISH for RNA foci detection was utilized, with 5 probes complementary to the CUG repeat region and 23 probes complementary to the last three exons of DMPK1 which are not composed of CUG repeats. UPF1 was down-regulated using siRNAs in DM1 and in normal fibroblasts and these cells were analyzed by SM-FISH 24 hours post siRNA-transfection. For both control fibroblasts and fibroblasts isolated from DM1 patients, UPF1 siRNAs decreased UPF1 protein levels by 35%-40% compared to scrambled siRNAs (FIGS. 13C and D). There was lower cell recovery after UPF1 knockdown, suggesting that knockdown of NMD components may cause a loss of cell viability, deflating the measured level of UPF1 knockdown. But even with the modest UPF1 knockdown, SM-FISH analysis revealed an increase in the number of nuclear foci in DM1 cells treated with UPF1 siRNAs compared to untreated DM1 cells or DM1 cells treated with mock siRNAs (FIG. 6A). In contrast, normal fibroblast cells bearing just a few CUG repeats in the DMPK gene exhibited no nuclear foci in both untreated or treated with UPF1 siRNAs (FIG. 6A). The number of foci present in the DM1 cells was quantified and UPF1 down-regulation caused a significant increase in the percentage of cells containing a higher number of foci (FIG. 6B). This data supports a conserved role for NMD in the identification of transcripts bearing GC-rich sequences in their 3′UTR. Furthermore, the results support the function of NMD as an important element in the toxicity of expanded CUG repeat transcripts in myotonic dystrophy 1.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A Caenorhabditis elegans (C. elegans) strain exhibiting an RNA toxicity phenotype, the strain comprising a detectable reporter gene expressed in one or more cell types, the expressed reporter gene RNA having an instance of at least fifty oligonucleotide repeats, optionally wherein the oligonucleotide repeats are repeats of from 3 to 6 nucleotides.

2. The C. elegans strain of claim 1, wherein the oligonucleotide repeats are trinucleotide repeats.

3. The C. elegans strain of claim 1, wherein the detectable reporter gene is stably integrated into the C. elegans genome.

4. The C. elegans strain of claim 1, wherein the C. elegans exhibits a decline in adult stage reporter gene protein levels.

5. The C. elegans strain of claim 1, wherein the reporter gene RNA accumulates into nuclear foci.

6. The C. elegans strain of claim 1, wherein the reporter gene is expressed from a tissue-specific promoter.

7. The C. elegans strain of claim 1, wherein the reporter gene is expressed in body wall muscle cells, and optionally wherein the C. elegans displays a motor defect in the adult stage.

8. The C. elegans strain of claim 1, wherein the reporter gene is expressed in neurons.

9. The C. elegans strain of claim 1, wherein the detectable reporter gene encodes a fluorescent or luminescent protein.

10. The C. elegans strain of claim 1, wherein the oligonucleotide repeats are in the 3′ UTR of the detectable reporter gene.

11. The C. elegans strain of claim 1, wherein the repeats are trinucleotide repeats that encode polyglutamine.

12. The C. elegans strain of claim 1, wherein the repeats are trinucleotide repeats of CUG, CGG or CAG.

13. The C. elegans strain of claim 1, wherein the reporter gene RNA has at least 70 repeats of the oligonucleotide, at least 100 repeats of the oligonucleotide, or at least 120 repeats of the oligonucleotide.

14. The C. elegans strain of claim 1, wherein the C. elegans strain further comprises an inactivation, overexpression, or modification of at least one endogenous gene, optionally wherein the endogenous gene encodes a signaling protein, a protein involved in RNA processing or degradation, RNA transport, transcription, DNA repair or recombination, or translation.

15. The C. elegans strain of claim 14, wherein the C. elegans strain comprises an inactivation of at least one endogenous gene by RNAi, optionally wherein the endogenous gene encodes a protein of the nonsense-mediated mRNA decay pathway and/or wherein the endogenous gene is a gene listed in Table 2 or 3.

16. A multiwell plate comprising a C. elegans strain of claim 1 in each of a plurality of wells.

17. The multiwell plate of claim 16, further comprising at least one well containing a C. elegans strain that does not exhibit an RNA toxicity phenotype, optionally wherein at least one C elegans strain that does not exhibit an RNA toxicity phenotype has a non-pathogenic amount of oligonucleotide repeats.

18. A method for determining an effect of an agent on an RNA toxicity phenotype, comprising:
providing the multiwell plate of claim 16,
adding a candidate agent to each of a plurality of wells,
quantifying an effect of the candidate agent on the RNA toxicity phenotype.

19. The method of claim 18, wherein the effect on the RNA toxicity phenotype is quantified by the level of protein expression of said reporter gene and/or cellular location of the reporter gene RNA, or by the accumulation of RNA into nuclear foci.

20. The method of claim 18, further comprising quantifying a change in motility.

21. The method of claim 18, comprising selecting an agent that reduces said RNA toxicity phenotype.

22. The method of claim 21, further comprising formulating the selected agent that reduces said RNA toxicity phenotype as a pharmaceutically acceptable composition, optionally wherein the agent is formulated for systemic administration.

23. The method of claim 21, wherein said agent inhibits or increases the expression or activity of a gene selected from Table 2 or 3, optionally wherein the gene is involved in the nonsense-mediated mRNA decay pathway.
